(19) United States
(12) Patent Application Publication
(10) Pub. No.: US 2004/0034078 A1
(43) Pub. Date: Feb. 19, 2004

(54) Benzimidazole inhibitors of poly(ADP-ribosyl) polymerase

(75) Inventors: Donald J. Skalitzky, San Diego, CA (US); Stephen E. Webber, San Diego, CA (US); Brian Eastman, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/453,973

(22) Filed: Jun. 4, 2003

Related U.S. Application Data
(60) Provisional application No. 60/388,840, filed on Jun. 14, 2002.

(51) Int. Cl. A61K 31/415; A61K 31/4184; C07D 235/04

(52) U.S. Cl. 514/394; 548/304.7

(57) ABSTRACT

Compounds of formula I are poly(ADP-ribosyl)transferase (PARP) inhibitors, and are useful as therapeutics in treatment of cancers and the amelioration of the effects of stroke, head trauma, and neurodegenerative disease. As cancer therapeutics, the compounds of the invention may be used in combination with cytotoxic agents and/or radiation.

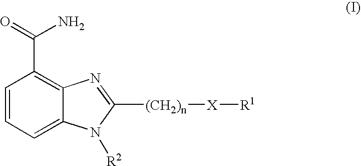

### CROSS-REFERENCE TO RELATED APPLICATION

This application claims the benefit of U.S. provisional application serial No. 60/388,840, filed Jun. 14, 2002, which is hereby incorporated by reference in its entirety.

### FIELD OF THE INVENTION

The invention pertains to agents that inhibit poly(ADP-ribose) polymerases, thereby retarding the repair of damaged DNA strands, and to processes of preparing such compounds. The invention also relates to the use of such compounds in pharmaceutical compositions and therapeutic treatments useful for potentiation of anti-cancer therapies, inhibition of neurotoxicity consequent to stroke, head trauma, and neurodegenerative diseases, and prevention of insulin-dependent diabetes.

### BACKGROUND OF THE INVENTION

Poly(ADP-ribose) polymerases (PARPs), nuclear enzymes found in almost all eukaryotic cells, catalyze the transfer of ADP-ribose units from nicotinamide adenine dinucleotide (NAD⁺) to nuclear acceptor proteins, and are responsible for the formation of protein-bound linear and branched homo-ADP-ribose polymers. Activation of PARP and resultant formation of poly(ADP-ribose) are induced by DNA strand breaks, e.g., after exposure to chemotherapy, ionizing radiation, oxygen free radicals, or nitric oxide (NO). The acceptor proteins of poly(ADP-ribose), including histones, topoisomerases, DNA and RNA polymerases, DNA ligases, and Ca²⁺ and Mg²⁺ dependent endonucleases, are involved in maintaining DNA integrity.

Because this cellular ADP-ribose transfer process is associated with the repair of DNA strand breakage in response to DNA damage caused by radiotherapy or chemotherapy, it can contribute to the resistance that often develops to various types of cancer therapies. Consequently, inhibition of PARP is thought to retard intracellular DNA repair and enhance the antitumor effects of cancer therapy. Indeed, in vitro and in vivo data show that many PARP inhibitors potentiate the effects of ionizing radiation or cytotoxic drugs such as DNA methylating agents. Therefore, inhibitors of the PARP enzyme are useful as adjunct cancer chemotherapeutics.

Ischemia, a deficiency of oxygen and glucose in a part of the body, can be caused by an obstruction in the blood vessel supplying that area or a massive hemorrhage. Two severe forms, heart attack and stroke, are major killers in the developed world. Cell death results directly and also occurs when the deprived area is reperfused. The development of PARP inhibitors to treat ischemia/reperfusion injuries has been reviewed by Zhang (Zhang, “PARP inhibition: a novel approach to treat ischaemia/reperfusion and inflammation-related injuries,” Emerging Drugs: The Prospect for Improved Medicines, Ashley Publications Ltd., 1999). Inhibition of PARP has been shown to protect against myocardial ischemia and reperfusion injury (Zingarelli et al., “Protection against myocardial ischemia and reperfusion injury by 3-aminobenzamide, an inhibitor of poly (ADP-ribose) synthetase,” Cardiovascular Research (1997), 36:205-215). Therefore, PARP inhibitors are a useful therapy in treating cardiovascular diseases.

After brain ischemia, the distribution of cells with accumulation of poly(ADP-ribose), that is, the areas where PARP has been activated, corresponds to the regions of ischemic damage (Love et al., “Neuronal accumulation of poly(ADP-ribose) after brain ischaemia,” Neuropathology and Applied Neurobiology (1999), 25:98-103). It has been shown that inhibition of PARP promotes resistance to brain injury after stroke (Endres et al., “Ischemic Brain Injury is Mediated by the Activation of Poly(ADP-Ribose)Polymerase,” J. Cerebral Blood Flow Metab. (1997), 17:1143-1151); Zhang, “PARP Inhibition Results in Substantial Neuroprotection in Cerebral Ischemia,” Cambridge Healthtech Institute 's Conference on Acute Neuronal Injury: New Therapeutic Opportunities, Sep. 18-24, 1998, Las Vegas, Nev.).

The activation of PARP by DNA damage is believed to play a role in the cell death consequent to head trauma and neurodegenerative diseases, as well as stroke. DNA is damaged by excessive amounts of NO produced when the NO synthase enzyme is activated as a result of a series of events initiated by the release of the neurotransmitter glutamate from depolarized nerve terminals (Cosi et al., “Poly(ADP-Ribose) Polymerase Revisited: A New Role for an Old Enzyme: PARP Involvement in Neurodegeneration and PARP Inhibitors as Possible Neuroprotective Agents,” Ann. N.Y. Acad. Sci. (1997); 825:366-379). Cell death is believed to occur as a result of energy depletion as NAD⁺ is consumed by the enzyme-catalyzed PARP reaction. Therefore, inhibitors of the PARP enzyme are useful inhibitors of neurotoxicity consequent to stroke, head trauma, and neurodegenerative diseases.

Parkinson's disease is an example of a neurodegenerative condition whose progression may be prevented by PARP inhibition. It has been demonstrated that mice that lack the gene for PARP are spared from the effects of exposure to 1-methyl-4-phenyl-1,2,3,6-tetrahydropyridine (MPTP), a neurotoxin that causes Parkinsonism in humans and animals (Mandir et al., “Poly(ADP-ribose) polymerase activation mediates 1-methyl-4-phenyl-1,2,3,6-tetrahydropyridine (MPTP)-induced Parkinsonism,” Proc. Natl. Acad. Sci. USA (1999), 96:5774-5779). MPTP activates PARP exclusively in dopamine-containing neurons of the substantia nigra, the part of the brain whose degeneration is associated with development of Parkinsonism. Hence, potent PARP inhibitors could slow the onset and development of this crippling condition.

Furthermore, inhibition of PARP should be a useful approach for treatment of conditions or diseases associated with cellular senescence, such as skin aging, through the role of PARP in the signaling of DNA damage. See, e.g., U.S. Pat. No. 5,589,483, which describes a method to extend the lifespan and proliferative capacity of cells comprising administering a therapeutically effective amount of a PARP inhibitor to the cells under conditions such that PARP activity is inhibited. Hence, inhibitors of the PARP enzyme are useful therapeutics for skin aging.

In yet a further application, PARP inhibition is being studied at the clinical level to prevent development of insulin-dependent diabetes mellitus in susceptible individuals (Saldeen et al., “Nicotinamide-induced apoptosis in insulin producing cells in associated with cleavage of poly(ADP-ribose) polymerase,” Mol. Cellular Endocrinol. (1998), 139:99-107). In models of Type I diabetes induced by toxins such as streptozocin and alloxan that destroy pancreatic islet cells, it has been shown that “knock-out” mice lacking PARP are resistant to cell destruction and diabetes development (see, e.g., Pieper et al., “Poly (ADP-ribose) polymerase, nitric oxide, and cell death,” Trends Pharmacolog. Sci. (1999), 20:171-181; see also Burkart et al., “Mice lacking the poly(ADP-ribose) polymerase gene are resistant to pancreatic beta-cell destruction and diabetes development induced by streptozocin,” Nature Medicine (1999), 5:314-319). Administration of nicotinamide, a weak PARP inhibitor and a free-radical scavenger, prevents development of diabetes in a spontaneous autoimmune diabetes model, the non-obese, diabetic mouse (Pieper et al., ibid.). Hence PARP inhibitors are useful as diabetes-prevention therapeutics.

PARP inhibition is also an approach for treating inflammatory conditions such as arthritis (Szabo et al., “Protective effect of an inhibitor of poly(ADP-ribose) synthetase in collagen-induced arthritis,” Portland Press Proc. (1998), 15:280-281; Szabo, “Role of Poly(ADP-ribose) Synthetase in Inflammation,” Eur. J. Biochem. (1998), 350(1):1-19; Szabo et al., “Protection Against Peroxynitrite-induced Fibroblast Injury and Arthritis Development by Inhibition of Poly(ADP-ribose) Synthetase,” Proc. Natl. Acad. Sci. USA (1998), 95(7):3867-72). PARP inhibitors are therefore useful as therapeutics for inflammatory conditions.

The PARP family of enzymes is extensive. It has recently been shown that tankyrases, which bind to the telomeric protein TRF-1, a negative regulator of telomere-length maintenance, have a catalytic domain that is strikingly homologous to PARP and have been shown to have PARP activity in vitro. It has been proposed that telomere function in human cells is regulated by poly(ADP-ribosyl)ation. PARP inhibitors have utility as tools to study this function. Further, as a consequence of regulation of telomerase activity by tankyrase, PARP inhibitors should have utility as agents for regulation of cell life-span, e.g., for use in cancer therapy to shorten the life-span of immortal tumor cells, or as anti-aging therapeutics, since telomere length is believed to be associated with cell senescence.

Competitive inhibitors of PARP are known. For example, Banasik et al. (“Specific Inhibitors of Poly(ADP-Ribose) Synthetase and Mono(ADP-Ribosyl) transferase,” J. Biol. Chem. (1992) 267:1569-1575) examined the PARP-inhibiting activity of certain compounds such as 4-amino-1,8-naphthalimide, 6(5H)-phenanthridone, 2-nitro-6(5H)-phenanthridone, and 1,5-dihydroxyisoquinoline. Griffin et al. reported the PARP-inhibiting activity for certain benzamide compounds (U.S. Pat. No. 5,756,510; see also “Novel Potent Inhibitors of the DNA Repair Enzyme Poly (ADP-ribose)polymerase (PARP),” Anti-Cancer Drug Design (1995), 10:507-514) and quinalozinone compounds (International Publication No. WO98/33802). Suto et al. (“Dihydroisoquinolines: The Design and Synthesis of a New Series of Potent Inhibitors of Poly(ADP-ribose) Polymerase,” Anti-Cancer Drug Design (1991), 7:107-117) reported PARP inhibition by certain dihydroisoquinoline compounds. Griffin et al. reported other PARP inhibitors of certain quinazolines (“Resistance-Modifying Agents. 5. Synthesis and Biological Properties of Quinazoline Inhibitors of the DNA Repair Enzyme Poly(ADP-ribose) Polymerase (PARP),” J. Med. Chem., ASAP Article 10.1021/jm980273t S0022-2623(98)00273-8; Web Release Date: Dec. 1, 1998). International Publication Nos. WO99/11622, WO99/11623, WO99/11624, WO99/11628, WO99/11644, WO99/11645, WO99/11649, WO00/29384, WO00/26192, and WO00/32579 describe certain PARP-inbibiting compounds. International Publication No. WO97/04771 describes certain benzimidazole-4-carboxamide compounds which act as PARP inhibitors. U.S. Pat. No. 5,756,510 also describes certain benzamide compounds useful as PARP inhibitors.

Certain heterocyclic compounds are also disclosed as being useful in the treatment of thrombic conditions and bone diseases. International Publication Nos. WO00/47573, WO99/06371, WO99/57113, and WO98/21188 disclose certain halogenated indole-, naphthalene-, benzimidazole-, and benzofuran-containing piperazine compounds which inhibit the activated coagulation protease Factor Xa. In addition, International Publication No. WO97/10219 discloses certain benzamidizole-containing compounds useful as inhibitors of V-type H⁺-ATPase, which is implicated in abnormal bone metabolism.

Nonetheless, there is still a need for small-molecule compounds that are PARP inhibitors and that have desirable or improved physical and chemical properties appropriate for pharmaceutical applications.

### SUMMARY OF THE INVENTION

The present invention is directed to agents that function as poly(ADPribosyl)transferase (PARP) inhibitors. The invention is also directed to the use of the agents as therapeutics, e.g., in treating cancers, inflammation, and diabetes and in ameliorating the effects of heart attack, stroke, head trauma, and neurodegenerative disease.

As cancer therapeutics, the compounds of the invention are used in a preferred embodiment in combination with DNA-damaging cytotoxic agents, such as methylating or strand breaking agents and/or radiation.

In one general aspect, the present invention is directed to compounds of the formula I:

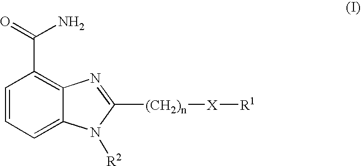

wherein:

n is 0 or 1; R¹ is H or an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together to cyclize to form part of a heteroaryl or heterocycloalkyl group unsubstituted or substituted with an unsubstituted alkyl group;

X is:

_S(O)ₘ—, wherein m is 0, 1, or 2; or

—N(R³)—, wherein R³ is H or C₁ to C₄ alkyl; or when n=1, —N(R³)— and R¹ together form a 3- to 10-membered heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more R_{c} groups together cyclize to form part of a heteroaryl or heterocycloalkyl group unsubstituted or substituted with an unsubstituted alkyl group; and

R² is H or alkyl;

or R¹ and R², together with the atoms to which they are bound, form a 5- to 8-membered heterocyclic ring unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

The invention is also directed to pharmaceutically acceptable salts, prodrugs, active metabolites, and solvates of compounds of formula I. Such compounds, salts, prodrugs, active metabolites and solvates are sometimes referred to herein as “PAPP-inhibiting agents.” Preferably, the PARP-inhibiting agents have an activity corresponding to a Kᵢ of 10 μM or less in a PARP enzyme inhibition assay.

The present invention is also directed to pharmaceutical compositions each comprising an effective PARP-inhibiting amount of a compound of formula I, or a pharmaceutically acceptable salt, prodrug, active metabolite, or solvate thereof together with a pharmaceutically acceptable carrier therefor.

The present invention is also directed to a method of inhibiting PARP enzyme activity, comprising contacting the enzyme with an effective amount of a compound of formula I.

The present invention is further directed to a method of potentiating the cytotoxicity of a cytotoxic drug or ionizing radiation, comprising contacting cells with an effective amount of a compound of formula I, or a pharmaceutically acceptable salt, prodrug, active metabolite, or solvate thereof, in combination with a cytotoxic drug or ionizing radiation. The PARP-inhibiting agents of the invention preferably have a cytotoxicity potentiation activity corresponding to a PF₅₀ of greater than 1 in a cytotoxicity potentiation assay.

The invention also provides methods useful in treating disease or an injury state where PARP activity is deleterious to a patient. The therapeutic methods each comprise inhibiting PARP enzyme activity in the relevant tissue of the patient by administering an effective amount of a compound of formula I, or a pharmaceutically acceptable salt, prodrug, active metabolite, or solvate thereof. In one preferred embodiment of such a method a cytotoxic drug and/or radiotherapy is administered to a mammal in conjunction with an effective PARP-inhibiting amount of a compound of formula I, or a pharmaceutically acceptable salt, prodrug, active metabolite, or solvate thereof.

A therapeutic method provided by the present invention is a cardiovascular therapeutic method for treating myocardial ischemia or reperfusion injury in a mammal, comprising administering to the mammal an effective amount of a compound of formula I, or a pharmaceutically acceptable salt, prodrug, active metabolite, or solvate thereof.

Another therapeutic method provided by the present invention is a method for treating neurotoxicity consequent to stroke, head trauma, or neurodegenerative disease in a mammal by administering an effective amount of a compound of formula I, or a pharmaceutically acceptable salt, prodrug, active metabolite, or solvate thereof, to the mammal.

Yet another therapeutic method provided by the present invention is for treating the onset of cell senescence associated with skin aging in a mammal, comprising administering to fibroblast cells in a mammal an effective PARP-inhibiting amount of a compound of formula I, or a pharmaceutically acceptable salt, prodrug, active metabolite, or solvate thereof.

Still a further therapeutic method provided by the present invention is a method to treat insulin-dependent diabetes mellitus in a susceptible individual, comprising administering to the individual an effective amount of a compound of formula I, or a pharmaceutically acceptable salt, prodrug, active metabolite, or solvate thereof.

The present invention also provides a therapeutic approach to treatment of inflammation, comprising administering an effective amount of a compound of formula I, or a pharmaceutically acceptable salt, prodrug, active metabolite, or solvate thereof, to a mammal in need of treatment.

The invention further relates to a process for preparing a compound of formula I wherein R¹ and R², together with the atoms to which they are bound, do not form a ring, the method comprising:

providing an electrophilic resin-bound precursor of formula II:

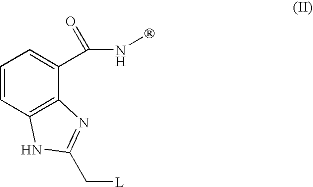

where L is a leaving group selected from the group consisting of Cl, Br, I, triflate, mesylate and tosylate;

reacting the electrophilic resin-bound precursor II with a suitable nucleophile R¹—X—H, where R¹ and X are as defined above and ® represents a support resin; and

cleaving the product from the resin to yield a compound of formula I.

Further preferred embodiments, features and advantages of the invention will become apparent from the following detailed description.

### DETAILED DESCRIPTION OF THE INVENTION AND PREFERRED EMBODIMENTS

PARP-Inhibiting Agents:

In accordance with a convention used in the art, the symbol

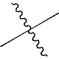

is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

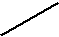

represents a methyl group,

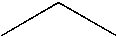

represents an ethyl group,

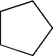

represents a cyclopentyl group, etc.

As used herein, the term “alkyl” means a branched- or straight-chained (linear) paraffinic hydrocarbon group (saturated aliphatic group) having from 1 to 10 carbon atoms in its chain, which may be generally represented by the formula CₖH₂ₖ₊₁, where k is an integer of from 1 to 10. Examples of alkyl groups include methyl, ethyl, n-propyl, isopropyl, n-butyl, isobutyl, t-butyl, pentyl, n-pentyl, isopentyl, neopentyl, and hexyl, and the simple aliphatic isomers thereof. A “lower alkyl” is intended to mean an alkyl group having from 1 to 4 carbon atoms in its chain.

The term “alkenyl” means a branched- or straight-chained olefinic hydrocarbon group (unsaturated aliphatic group having one or more double bonds) containing 2 to 10 carbons in its chain. Exemplary alkenyls include ethenyl, 1-propenyl, 2-propenyl, 1-butenyl, 2-butenyl, isobutenyl, and the various isomeric pentenyls and hexenyls (including both cis and trans isomers).

The term “alkynyl” means a branched or straight-chained hydrocarbon group having one or more carbon-carbon triple bonds, and having from 2 to 10 carbon atoms in its chain. Exemplary alkynyls include ethynyl, propynyl, 1-butynyl, 2-butynyl, and 2-pentynyl.

The term “carbocycle” refers to a saturated, partially saturated, unsaturated, or aromatic, monocyclic or fused or non-fused polycyclic, ring structure having only carbon ring atoms (no heteroatoms, i.e., non-carbon ring atoms). Exemplary carbocycles include cycloalkyl, aryl, and cycloalkyl-aryl groups.

The term “heterocycle” refers to a saturated, partially saturated, unsaturated, or aromatic, monocyclic or fused or non-fused polycyclic, ring structure having one or more heteroatoms selected from N, O, and S. Exemplary heterocycles include heterocycloalkyl, heteroaryl, and heterocycloalkyl-heteroaryl groups.

A “cycloalkyl group” is intended to mean a non-aromatic monovalent, monocyclic or fused polycyclic, ring structure having a total of from 3 to 18 carbon ring atoms (but no heteroatoms). Exemplary cycloalkyls include cyclopropyl, cyclobutyl, cyclopentyl, cyclopentenyl, cyclohexyl, cycloheptyl, adamantyl, and like groups.

A “heterocycloalkyl group” is intended to mean a non-aromatic monovalent, monocyclic or fused polycyclic, ring structure having a total of from 3 to 18 ring atoms, including 1 to 5 heteroatoms selected from nitrogen, oxygen, and sulfur. Illustrative examples of heterocycloalkyl groups include pyrrolidinyl, tetrahydrofuryl, piperidinyl, piperazinyl, morpholinyl, thiomorpholinyl, aziridinyl, and like groups.

The term “aryl” means an aromatic monocyclic or fused polycyclic ring structure having a total of from 4 to 18 ring carbon atoms (no heteroatoms). Exemplary aryl groups include phenyl, naphthyl, anthracenyl, and the like.

A “heteroaryl group” is intended to mean an aromatic monovalent, monocyclic or fused polycyclic, ring structure having from 4 to 18 ring atoms, including from 1 to 5 heteroatoms selected from nitrogen, oxygen, and sulfur. Illustrative examples of heteroaryl groups include pyrrolyl, thienyl, oxazolyl, pyrazolyl, thiazolyl, furyl, pyridinyl, pyrazinyl, triazolyl, tetrazolyl, indolyl, quinolinyl, quinoxalinyl, and the like.

A “PARP-inhibiting agent” means a compound represented by formula I or a pharmaceutically acceptable salt, prodrug, active metabolite or solvate thereof.

A “prodrug” is a compound that may be converted under physiological conditions or by solvolysis to the specified compound or to a pharmaceutically acceptable salt of such compound. An “active metabolite” is a pharmacologically active product produced through metabolism in the body of a specified compound or salt thereof. Prodrugs and active metabolites of a compound may be identified using routine techniques known in the art. See, e.g., Bertolini et al., J. Med. Chem., (1997) 40:2011-2016; Shan et al., J. Pharm. Sci., 86 (7):765-767; Bagshawe, Drug Dev. Res., (1995) 34:220-230; Bodor, Advances in Drug Res., (1984) 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B, (2000) 748:281-293; Spraul et al., J. Pharmaceutical & Biomedical Analysis, (1992) 10 (8):601-605; and Prox et al., Xenobiol, (1992) 3 (2):103-112.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine. A “pharmaceutically acceptable salt” is intended to mean a salt that retains the biological effectiveness of the free acids and bases of the specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

If an inventive compound is a base, a desired salt may be prepared by any suitable method known to the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid; amino acid, such as aspartic acid or glutamic acid; aromatic acid, such as benzoic acid or cinnamic acid, sulfonic acid, such as p-toluenesulfonic acid or ethanesulfonic acid, and the like.

If an inventive compound is an acid, a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of compounds, salts, or solvates that are solids, it is understood by those skilled in the art that the inventive compounds, salts, and solvates may exist in different polymorph or crystal forms, all of which are intended to be within the scope of the present invention and specified formulas.

In some cases, the inventive compounds will have chiral centers. When chiral centers are present, the inventive compounds may exist as single stereoisomers, racemates, and/or mixtures of enantiomers and/or diastereomers. All such single stereoisomers, racemates, and mixtures thereof are intended to be within the broad scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

In one of its preferred aspects, the present invention is directed to compounds of formula I-a:

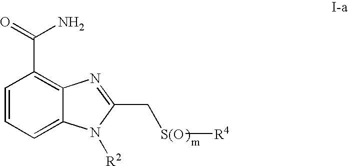

wherein:

R² is H or alkyl; and

R⁴ is hydrogen or an alkyl, aryl, heteroaryl, or heterocycloalkyl group unsubstituted or substituted with one or more substituents selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR, —NR_{c}R_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In another preferred aspect, the invention is directed to compounds of the formula I-b:

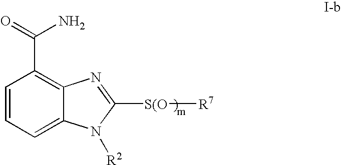

wherein:

R² is H or alkyl; and

R⁷ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, and —NO₂, and alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NFR_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In a further preferred aspect, the compounds are represented by the formula I-z:

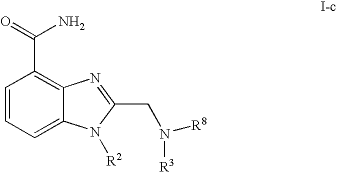

wherein:

R² is H or alkyl; and

R⁸ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, and —NO₂, and alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group,

or R³ and R⁸, together with the atoms to which they are bound, form a 3- to 10-membered heterocyclic ring unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

In yet another aspect, compounds of the invention have formula I-d:

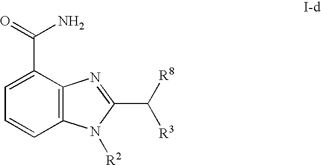

where R², R³ and R⁸ are defined above in connection with formula I-c.

Exemplary compounds of the invention represented by formula I include:

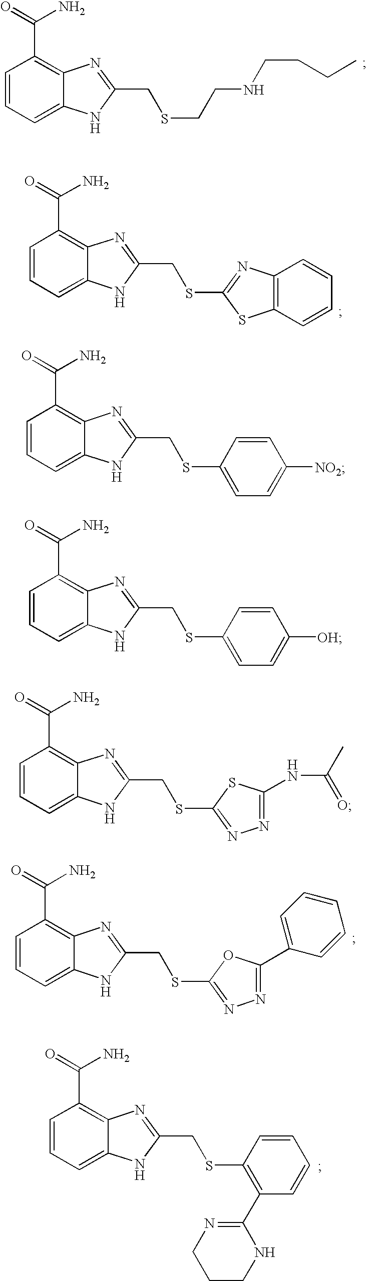

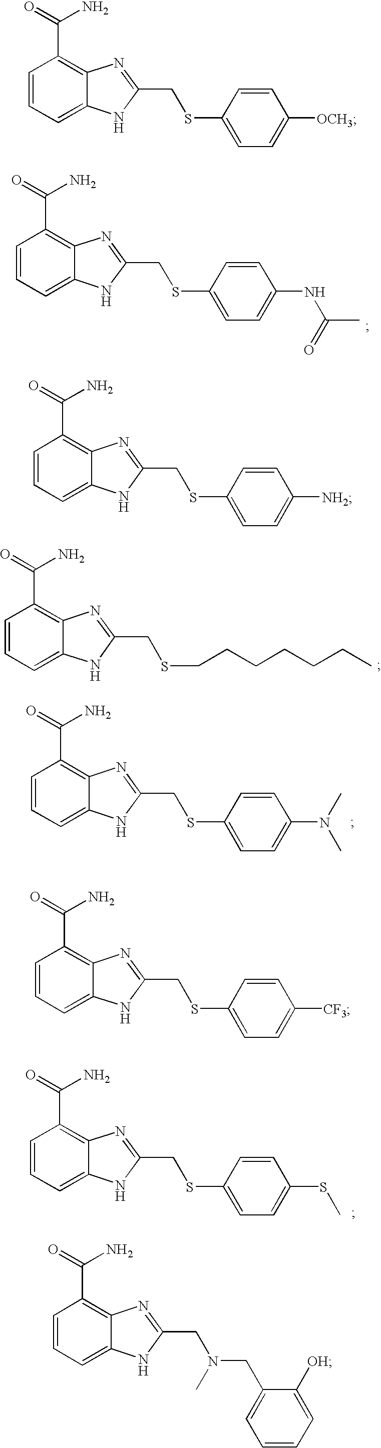

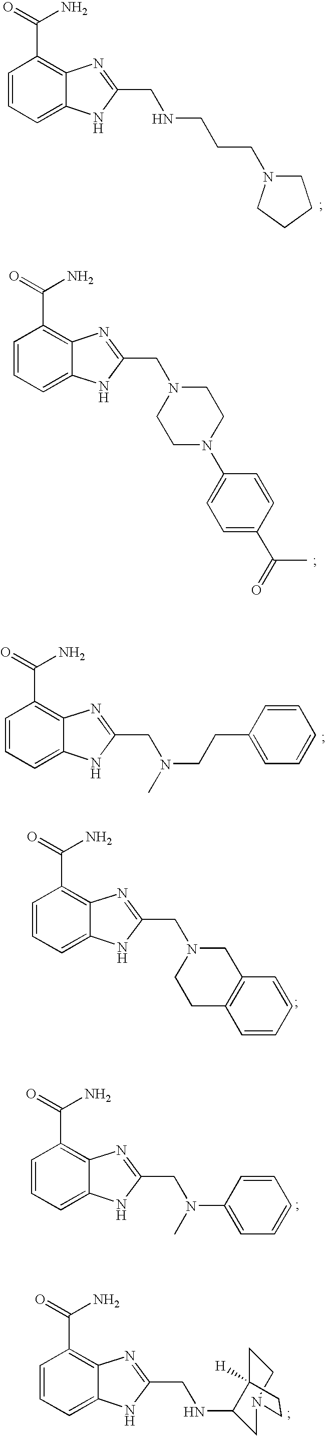

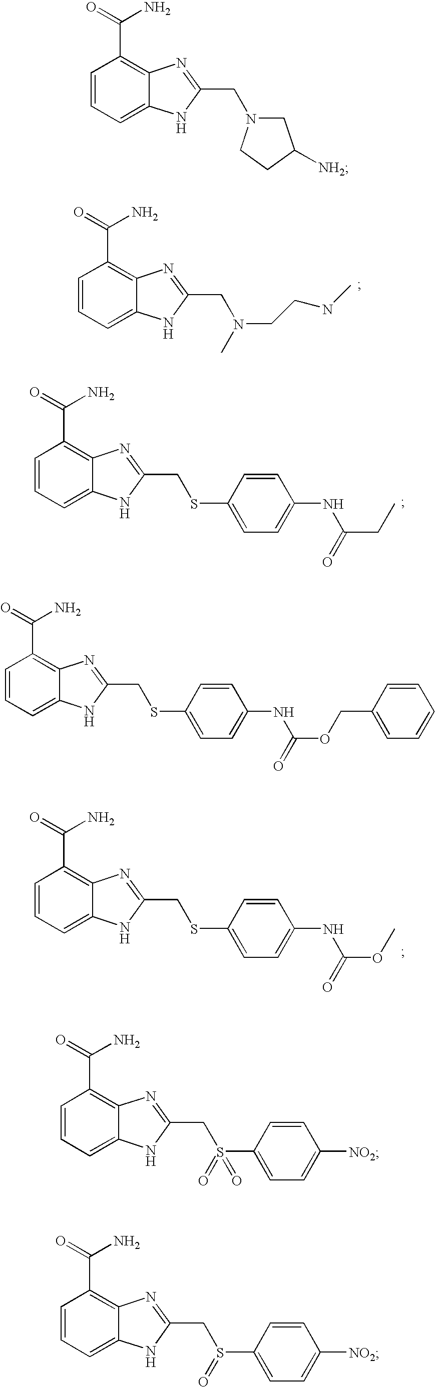

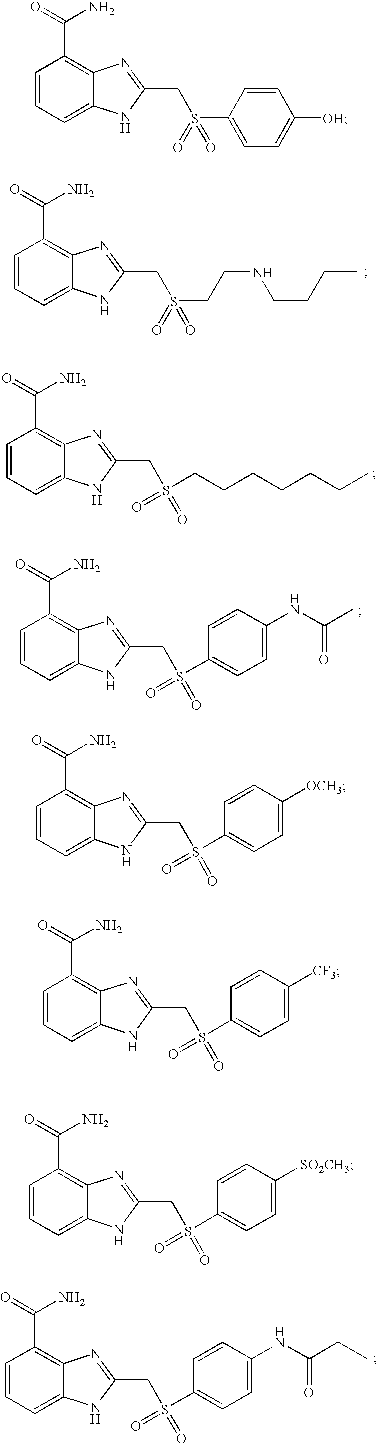

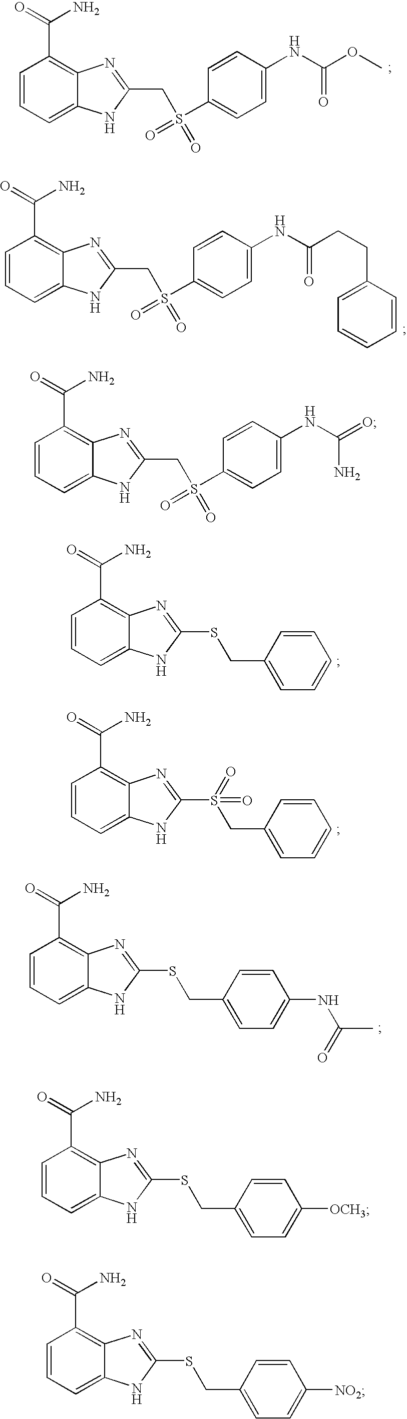

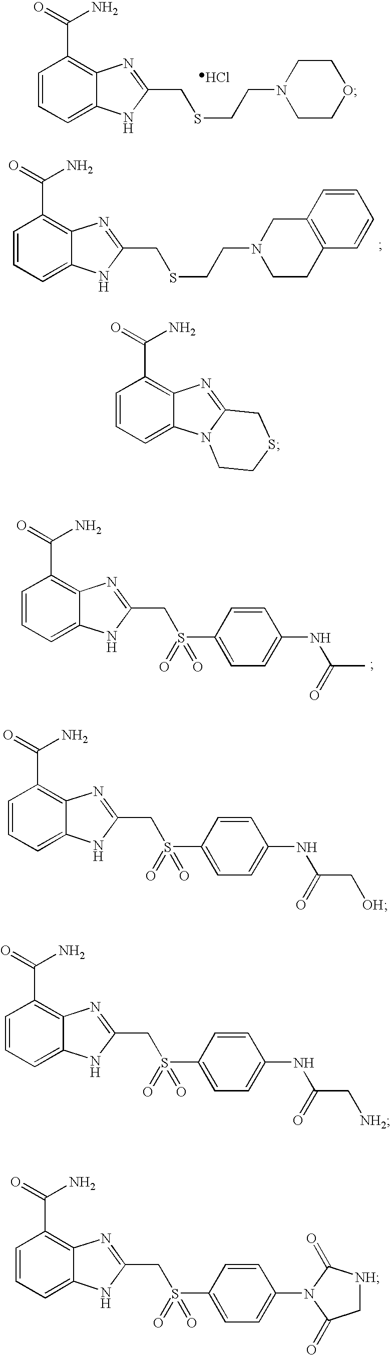

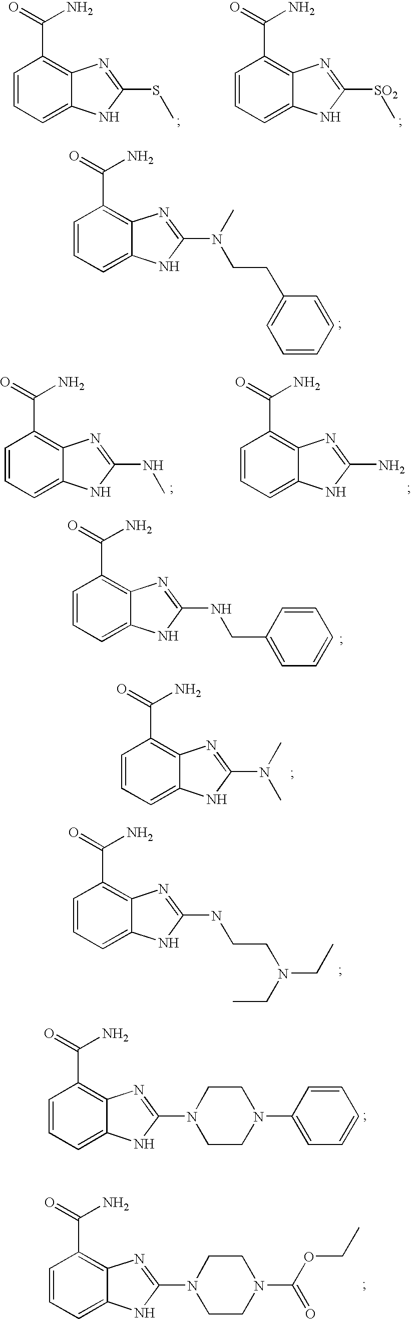

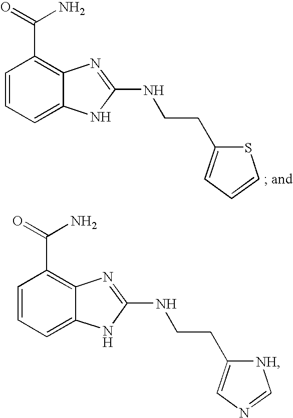

and pharmaceutically acceptable salts, prodrugs, active metabolites, and solvates thereof.

Exemplary compounds of the invention of formula I include:

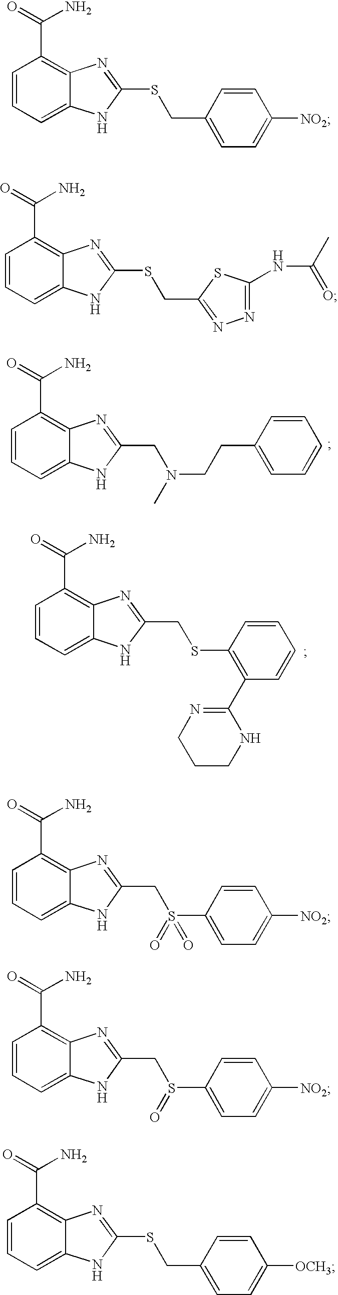

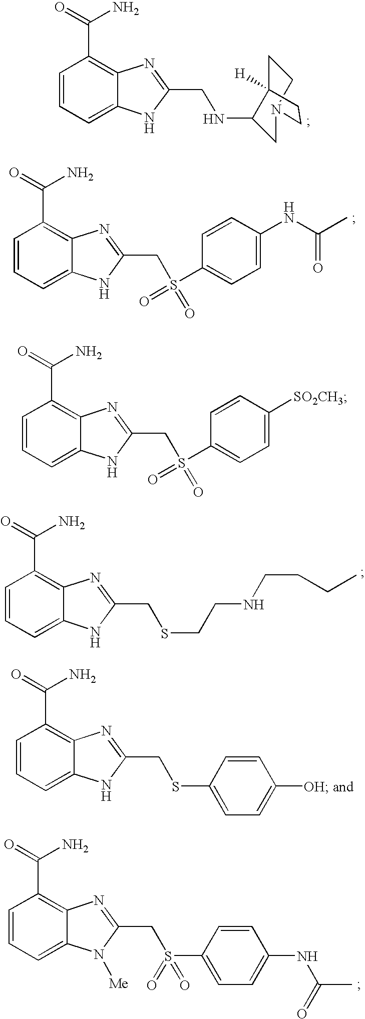

and pharmaceutically acceptable salts and solvates thereof.

The present invention is also directed to a method of inhibiting PARP enzyme activity, comprising contacting the enzyme with an effective amount of a compound of formula T, or a pharmaceutically acceptable salt, prodrug, pharmaceutically active metabolite, or solvate thereof. For example, PARP activity may be inhibited in mammalian tissue by administering a PARP-inhibiting agent according to the invention.

“Treating” or “treatment” is intended to mean at least the mitigation of an injury or a disease condition in a mammal, such as a human, that is alleviated by the inhibition of PARP activity, such as for potentiation of anti-cancer therapies or inhibition of neurotoxicity consequent to stroke, head trauma, or neurodegenerative diseases; and includes: (a) prophylactic treatment in a mammal, particularly when the mammal is found to be predisposed to having the disease condition but not yet diagnosed as having it; (b) inhibiting the disease condition; and/or (c) alleviating, in whole or in part, the disease condition.

The activity of the inventive compounds as inhibitors of PARP activity may be measured by any of the suitable methods available in the art, including in vivo and in vitro assays. An example of a suitable assay for activity measurements is the PARP enzyme inhibition assay described herein.

Administration of the compounds of the formula I and their pharmaceutically acceptable prodrugs, salts, active metabolites, and solvates may be performed according to any of the accepted modes of administration available to those skilled in the art. Illustrative examples of suitable modes of administration include oral, nasal, parenteral, topical, transdermal, and rectal. Oral and intravenous delivery are preferred.

A PARP-inhibiting agent may be administered as a pharmaceutical composition in any suitable pharmaceutical form. Suitable pharmaceutical forms include solid, semisolid, liquid, or lyopholized formulations, such as tablets, powders, capsules, suppositories, suspensions, liposomes, and aerosols. The PARP-inhibiting agent may be prepared as a solution using any of a variety of methodologies. For example, the PARP-inhibiting agent can be dissolved with acid (e.g., 1 M HCl) and diluted with a sufficient volume of a solution of 5% dextrose in water (D5W) to yield the desired final concentration of PARP-inhibiting agent (e.g., about 15 mM). Alternatively, a solution of D5W containing about 15 mM HCl can be used to provide a solution of the PARP-inhibiting agent at the appropriate concentration. Further, the PARP-inhibiting agent can be dissolved in ethanol and mixed with Cremophor® EL (polyoxyl 35 castor oil; BASF AKTIENGESELLSCHAFT CORP.). The ethanol can then be removed by drying with nitrogen and the desired concentration of PARP-inhibiting agent obtained by diluting the solution with D5W. Still further, the PARP-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition contains at least a therapeutically effective amount of the PARP-inhibiting agent and preferably is made up of one or more pharmaceutical dosage units. The selected dose may be administered to a mammal, for example, a human patient, in need of treatment mediated by inhibition of PARP activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

The phrases “therapeutically effective amount” and “effective amount” are intended to mean the amount of an inventive agent that, when administered to a mammal in need of treatment, is sufficient to effect treatment for injury or disease conditions alleviated by the inhibition of PARP activity, such as for potentiation of anti-cancer therapies or inhibition of neurotoxicity consequent to stroke, head trauma, and neurodegenerative diseases. The amount of a given compound of the invention that will be therapeutically effective will vary depending upon factors such as the particular compound, the disease condition and the severity thereof, the identity and characteristics of the mammal in need thereof, which amount may be routinely determined by artisans.

It will be appreciated that the actual dosages of the PARP-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals.

Synthetic Methods:

The compounds according to the invention may be advantageously prepared as set out in the examples below.

The structures of the compounds of the following examples were confirmed by one or more of the following: proton magnetic resonance spectroscopy, infrared spectroscopy, elemental microanalysis, mass spectrometry, thin layer chromatography, melting point, boiling point, and HPLC.

Proton magnetic resonance (¹H NMR) spectra were determined using a 300 megahertz Tech-Mag, Bruker Avance 300DPX, or Bruker Avance 500 DRX spectrometer operating at a field strength of 300 or 500 megahertz (MHz). Chemical shifts are reported in parts per million (ppm, δ) downfield from an internal tetramethylsilane standard. Alternatively, ¹H NMR spectra were referenced to residual protic solvent signals as follows: CHCl₃=7.26 ppm; DMSO=2.49 ppm; C6HD5=7.15 ppm. Peak multiplicities are designated as follows: s=singlet; d=doublet; dd=doublet of doublets; t=triplet; q=quartet; br=broad resonance; and m=multiplet. Coupling constants are given in Hertz. Infrared absorption (IR) spectra were obtained using a Perkin-Elmer 1600 series FTIR spectrometer. Elemental microanalyses were performed by Atlantic Microlab Inc. (Norcross, Ga.) and gave results for the elements stated within ±0.4% of the theoretical values. Flash column chromatography was performed using Silica gel 60 (Merck Art 9385). Analytical thin layer chromatography (TLC) was performed using precoated sheets of Silica 60 F₂₅₄ (Merck Art 5719). HPLC chromatographs were run on a Hewlett Packard Model 1100 system fitted with a Zorbax SB-C18 4.6 mm×150 mm column having 3.5 micron packing material. Unless otherwise stated, a ramp of 5% CH₃CN/H₂O to 95% CH₃CN/H₂O over 7.5 minutes then holding at 95% CH₃CN/H₂O for 2.5 minutes (both solvents contained 0.1% v/v TFA) at a flow of 1 mL/min was used. Retention times (Rt) are given in minutes. Semi-preparative HPLC were run on a Gilson LC3D system fitted with a 21.2 mm×250 mm C8 column. Ramps were optimized for each compound with a CH₃CN/H₂O solvent system. Melting points (abbreviated as mp) were determined on a Mel-Temp apparatus and are uncorrected. All reactions were performed in septum-sealed flasks under a slight positive pressure of argon, unless otherwise noted. All commercial reagents were used as received from their respective suppliers with the following exceptions: tetrahydrofuran (THF) was distilled from sodium-benzophenone ketyl prior to use; dichloromethane (CH₂Cl₂) was distilled from calcium hydride prior to use; anhydrous lithium chloride was prepared by heating at 110° C. under vacuum overnight. Mass spectra, both low and high resolution, were measured using either electrospray (EI) or fast atom bombardment (FAB) ionization techniques.

The following abbreviations are used herein: Et₂O (diethyl ether); DMF (N,N-dimethylformamide); DMSO (dimethylsulfoxide); MeOH (methanol); EtOH (ethanol); EtOAc (ethyl acetate); Ac (acetyl); Me (methyl); Et (ethyl); Ph (phenyl); DIEA (diisopropylethylamine); TFA (trifluoroacetic acid); HATU (O-(7-azabenzotriazol-1-yl)-N,N,N′,N′-tetramethyluronium hexafluorophosphate); DBU (1,8-diazabicyclo[5.4.0]undec-7-ene); TFFH (tetramethylfluoroformamidinium hexafluorophosphate).

Solid-phase syntheses were performed by immobilizing reagents with Rink amide linkers (Rink, Tetrahedron Letters (1987) 28:3787), which are standard acid-cleavable linkers that upon cleavage generate a free carboxamide group. Small-scale solid-phase syntheses, e.g., about 2-5 μmole, were performed using Chiron SynPhase® polystyrene O-series crowns (pins) derivatized with Fmoc-protected Rink amide linkers. For larger scale (e.g., greater than about 100 μmole) syntheses, the Rink amide linkages were formed to Argonaut Technologies Argogel® resin, a grafted polystyrene-poly(ethylene glycol) copolymer. Any suitable resin may be used as the solid phase, selected from resins that are physically resilient and that, other than with regard to the linking and cleavage reactions, are inert to the synthetic reaction conditions.

The following general approach can be used to prepare the compounds of the invention:

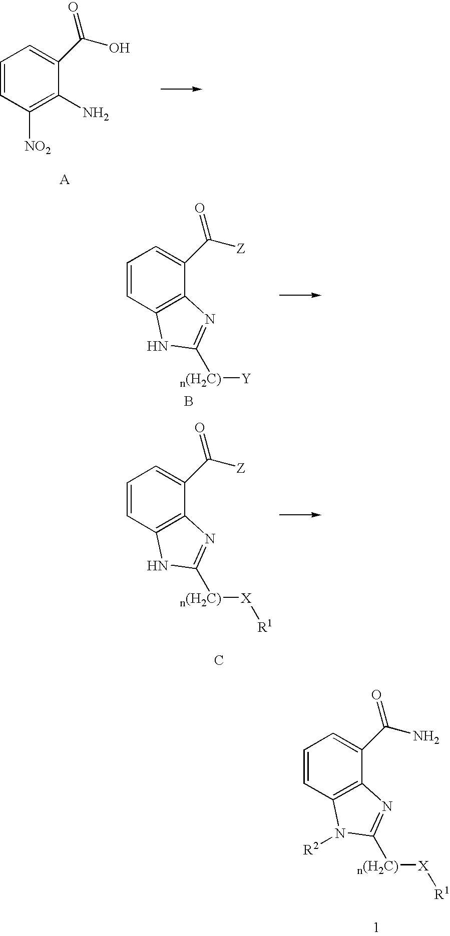

3-Nitroanthranilic acid A (3-amino-2-nitro-benzoic acid) is converted to a benzylic electrophile B (Y=Cl, n=1 and Z=ester, amide or resin-bound amide), which is alkylated by a nucleophile to give C (X═N or S). This intermediate is then converted to I by further modification of —XR¹ and/or ring nitrogen (R²) and conversion to the free primary carboxamide. Alternatively, A is converted to a nucleophile B (n=0, Y═SH and Z=ester, amide or resin-bound amide), which is then alkylated to give intermediate C. Conversion of C to the primary carboxamide gives I, which may undergo additional functionalization.

The following reaction schemes are useful in preparing compounds of the invention.

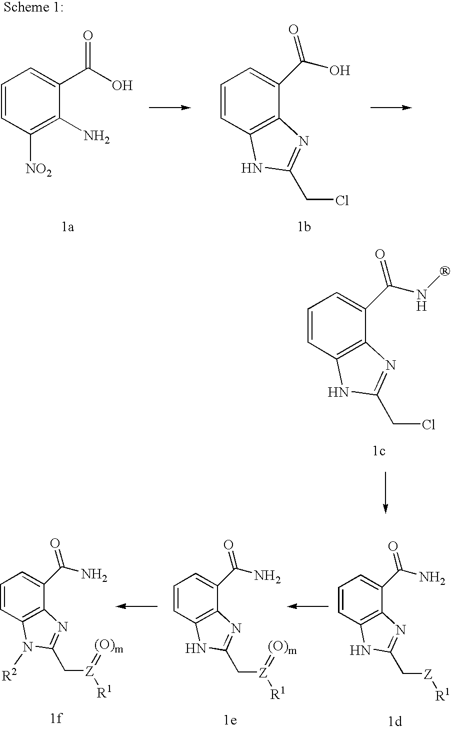

In Scheme 1, 3-nitroanthranilic acid (1a) is converted to the chloromethyl derivative 1b. This compound is then coupled to an appropriately functionalized solid support to give intermediate 1c. This material is treated with a nucleophile to displace the chloride to give 1d (Z═N or S, R¹=alkyl, aryl, etc.), after cleavage from the solid support. When Z═S the compound may be further transformed by oxidation with Oxone, hydrogen peroxide, potassium permanganate or similar reagent to a sulfoxide or sulfone (m=1 or 2, respectively). Additional modifications can be made by substitution on the benzimidazole core to give 1f. In all cases, 1d, 1e or 1f are optionally modified at R¹ or R².

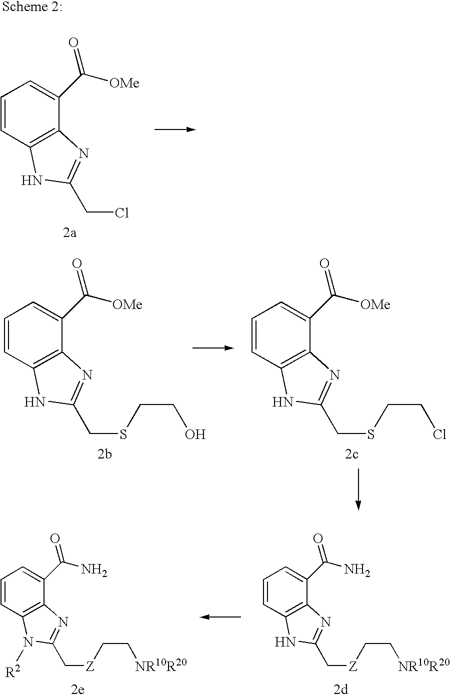

In Scheme 2, chloride 2a is alkylated with mercaptoethanol to give alcohol 2b. The alcohol is converted to chloride 2c by reaction with thionyl chloride or similar reagent. This intermediate is then alkylated with an appropriate amine (HNR¹⁰R²⁰) to give product 2d (Z═S). Compound 2b may be oxidized at sulfur to a sulfoxide [Z═S(O)] or sulfone [Z═(O)₂], and/or further modified at ring nitrogen (R²). In all cases, 2d and 2e are optionally modified at R², R¹⁰ or R²⁰.

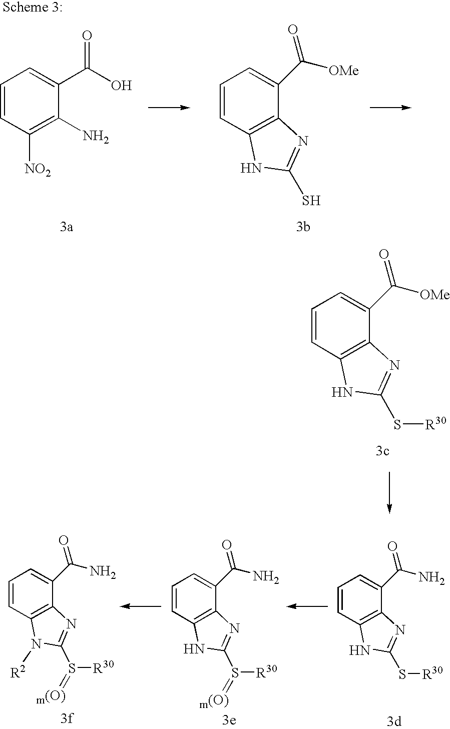

In Scheme 3, 3-nitroanthranilic acid is esterified, reduced and cyclized to intermediate 3b. Alkylation using cesium carbonate, or an equivalent base, and an appropriate electrophile give product of the type 3c. The ester is converted either directly to the amide 3d via the method of Jagdmann et. al. (Synth. Commun. (1990) 20:1203-1208, preferred method) or by a standard three-step method (ester hydrolysis, acid chloride formation and treatment with ammonia). Product 3d may be further modified by oxidation of sulfur (m=1 or 2) and/or substitution on the aromatic core (R²). In all cases, 3d, 3e or 3f may also be further derivatized, 1f desired.

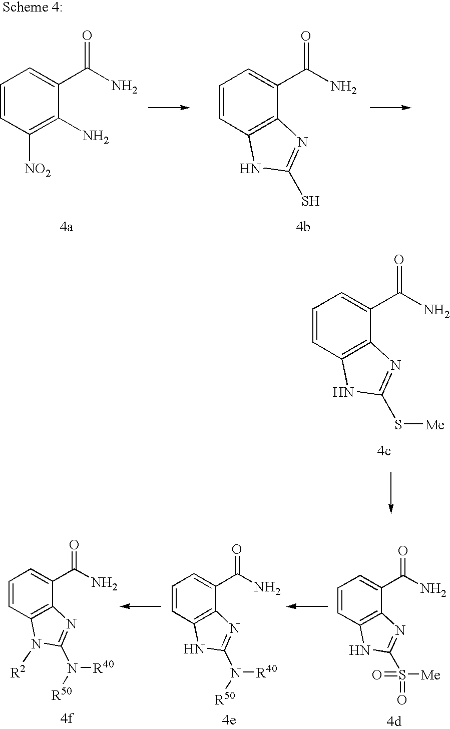

In Scheme 4, 2-amino-3-nitro-benzamide (4a) is reduced and cyclized with thiocarbonyl diimidazole to give thiourea derivative 4b. Alkylation with methyl iodide produces thioether 4c. Sulfur oxidation with Oxone, m-chloroperbenzoic acid, or a similar reagent gives advanced intermediate 4d. Nucleophileic substitution of sulfone 4d gives the desired 2-amino benzimidazole 4e. Modification of the benzimidazole core gives 4f. In all cases, 4e or 4f may be optionally modified at R⁴⁰ or R⁵⁰.

### EXAMPLES

### Example 1

### 2-(2-Butylamino-ethylsulfanylmethyl)-1H-benzimidazole-4-carboxylic Acid Amide (1)

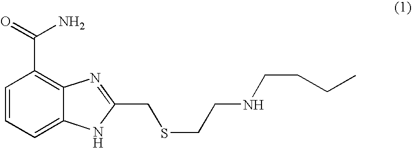

(a) 2-Chloromethyl-1H-benzimidazole-4-carboxylic Acid

2-Amino-3-nitro-benzoic acid (5.71 g, 31.4 mmol) was dissolved in 100 mL of methanol and a slurry of 10% Palladium on carbon (0.50 g) in 25 mL of methanol was then added. The reaction was stirred under H₂ atmosphere at 23° C. for 3 hr. The reaction mixture was filtered through Celite® media and the solvent removed in vacuo. Aqueous HCl (4N, 100 mL) was then added, followed by chloroacetic acid (8.90 g, 94.2 mmol) and the reaction was refluxed for 2.5 hr. The reaction was concentrated and the resulting black solid was then dissolved in 200 mL of boiling methanol. To this solution was added 4 g of activated charcoal. After 15 minutes the solution was filtered through Celite® and the filtrate was concentrated in vacuo to give 6.14 g (93%) of a red amorphous solid, which was used in the next step without further purification.

IR (KBr) 3504, 2962, 1728, 1631, 1253, 1199 cm⁻¹; ¹H NMR (DMSO-d₆) δ 5.0146 (s, 2H), 7.46 (t, 1H, J=7.7 Hz), 7.95 (d, 1H, J=7.7 Hz), 7.98 (d, 1H, J=7.7 Hz).

(b) 2-Chloromethyl-1H-benzimidazole-4-carboxylic Acid Amide Resin (“resin”)

To 2-chloromethyl-1H-benzimidazole-4-carboxylic acid (0.63 g, 2.95 mmol) was added 10 mL of thionyl chloride. The reaction was heated to reflux for 2 hr, cooled to 23° C. and concentrated by vacuum distillation. The crude 2-chloromethyl-1H-benzimidazole-4-carbonyl chloride was dissolved in 5% DIEA/CH₂Cl₂ (60 mL) and added to ArgoGel® poly(ethylene glycol) grafted polystyrene Rink amide functionalized resin (6.0 g, 0.33 mmol/g) prepared as described previously (Rink, Tetrahedron Letters (1987) 28:3787). The resin had the Fmoc protecting group removed by a 30 min treatment with 1% DBU in CH₂Cl₂. The acylated resin was filtered and washed consecutively with 50 mL CH₂Cl₂, DMF, CH₂Cl₂, DMF, CH₂Cl₂, CH₂Cl₂ and dried under vacuum for 24 hr. A small sample of resin was checked by cleavage with 95% TFA/H₂O for 30 min, followed HPLC and MS analysis. Throughout the following experimental protocols, the product material is referred to as “resin.”

HPLC Rt=4.10 min., MS calcd for C₉H₈N₃O₁+H 210/212 found 210/212.

(c) 2-(2-Butylamino-ethylsulfanylmethyl)-1H-benzimidazole-4-carboxylic Acid Amide (1)

2-Butylamino-ethanethio](0.30 g, 2.25 mmol) was added to 0.20 g of 2-chloromethyl-1H-benzimidazole-4-carboxylic acid amide resin suspended in 5% DIEA/DMF (5 mL). The reaction was shaken in a wrist action shaker for 12 hr and then filtered and washed as described above. The product was cleaved by treatment with 5 mL of 95% TFA/H₂O for 2 hr. The resin was filtered and rinsed with addition TFA/H₂O. The combined filtrates were reduced in vacuo and the residue purified by flash silica gel chromatography using a solvent system (5% MeOH/EtOAc) to give 15 mg of a white amorphous solid.

¹H NMR (DMSO-d₆) δ 1.33 (t, 3H, J=7.0 Hz), 1.65-2.03 (m, 5H), 3.15 (t, 2H, J=7.0 Hz), 3.31-3.43 (m, 4H), 4.55 (s, 2H), 7.28 (s, 1H), 7.63-7.79 (m, 2H), 8.12 (d, 1H, J=7.7 Hz), 8.38 (d, 1H, J=7.7 Hz), 9.68 (s, 1H); HPLC Rt=4.22 min. HRMS calcd for C₁₅H₂₂N₄O₁S₁+H 307.1598, found 307.1603.

The compounds of Examples 2-23 were prepared in the manner described for Example 1, except with varying the nucleophile in step 1(c), e.g.:

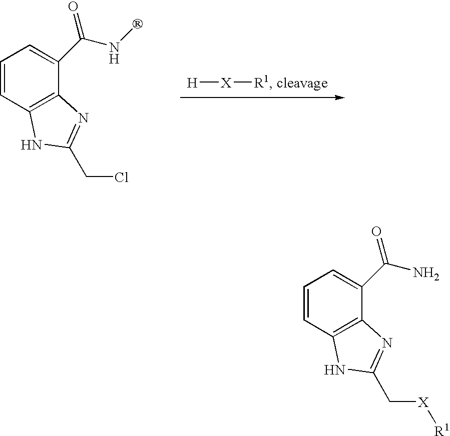

### Example 2

### 2-(Benzothiazol-2-ylsulfanylmethyl)-1H-benzimidazole-4 carboxylic Acid Amide (2)

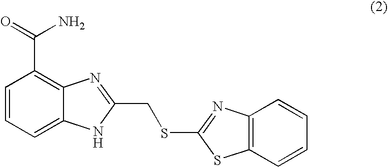

IR (KBr) 3375, 3175, 1662, 1604, 1535, 1496, 1423, 1309, 1242, 1006, 752 cm⁻¹; ¹H NMR (Acetone-d₆) (mixture of rotamers, 12H) δ 4.92 (s), 5.04 (s), 6.83 (br s), 7.25 (t, J=7.7 Hz), 7.32 (t, J=7.7 Hz), 7.35-7.58 (m), 7.68 (dd, J=1.1, 7.7 Hz), 7.80 (dd, J=2.2, 8.1 Hz), 7.97-8.00 (m), 8.24 (d, J=8.1 Hz), 9.37 (br s), 12.0 (br s), 12.49 (br s). HRMS calcd for C₁₆H₁₂N₄OS₂+H 341.0531, found 341.0545.

### Example 3

### 2-(4-Nitro-phenylsulfanylmethyl)-1H-benzimidazole-4-carboxylic Acid Amide (3)

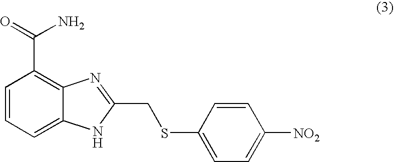

IR (KBr) 3406, 1674, 1599, 1581, 1340, 1205, 1140 cm⁻¹; ¹H NMR (DMSO-d₆) δ 5.24 (s, 2H), 7.27 (s, 1H), 7.74 (t, 1H, J=7.7 Hz), 8.13 (d, 1H, J=7.7 Hz), 8.19 (d, 2H, J=8.8 Hz), 8.37 (d, 1H, J=7.7 Hz), 8.60 (d, 2H J=8.8 Hz), 9.08 (br s, 1H), 11.99 (br s, 1H); HPLC Rt=5.55 min. MS calcd for C₁₅H₁₂N₄O₃S₁+H 329 found 329.

### Example 4

### 2-(4-Hydroxy-phenylsulfanylmethyl)-1H-benzimidazole-4-carboxylic Acid Amide (4)

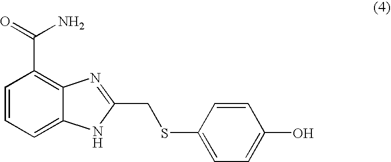

IR (KBr) 3377, 1657, 1589, 1583, 1425, 1205, 831, 756 cm⁻¹; ¹H NMR (DMSO-d₆) δ 2.72 (s, 1H), 4.32 (s, 2H), 6.67 (d, 2H), J=8.8 Hz), 7.20-7.29 (m, 3H), 7.64-7.67 (m, 2H), 7.78 (d, 1H, J=7.7 Hz), 7.94 (br s, 1H), 9.64 (br s, 1H); HPLC Rt=4.54 min. HRMS calcd for C₁₅H₁₃N₃O₂S₁+H 300.0807, found 300.0817. Anal. (C₁₅H₁₃N₃O₂S₁.0.8 H₂O.0.5 EtOAc.0.45 TFA) C, H, N, S.

### Example 5

### 2-(5-Acetylamino-[1,3,4]thiadiazol-2-ylsulfanylmethyl)-1H-benzimidazole-4-carboxylic Acid Amide (5)

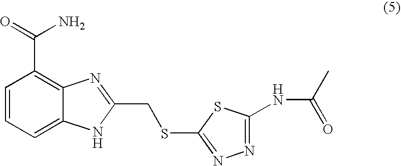

IR (KBr) 3356, 1680, 1606, 1207, 1132, 723 cm⁻¹; ¹H NMR (DMSO-d₆) (mixture of rotamers, 12H) δ 2.2 (s), 4.83 (s), 7.17-7.32 (m), 7.52 (br s), 7.66-7.82 (m), 8.15 (br s), 8.15 (br s), 9.07 (br s), 12.67 (br s), 13.07 (br s); HPLC Rt=4.48 min. HRMS calcd for C₁₃H₁₂N₆O₂S₂+Na 371.0361 found 371.0352. Anal. (C₁₃H₁₂N₆O₂S₂.1.0 H₂O) C, H, N, S.

### Example 6

### 2-(5-Phenyl-[1,3,4]oxadiazol-2-ylsulfanylmethyl)-1H-benzimidazole-4-carboxylic Acid Amide (6)

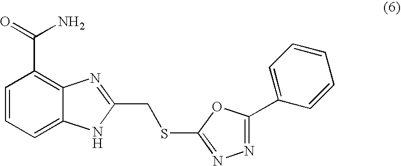

¹H NMR (DMSO-d₆) δ 4.87 (s, 2H), 7.28 (m, 1H), 7.53-7.64 (m, 5H), 7.80 (d, 1H, J=7.8 Hz), 7.91 (d, 2H, J=7.4 Hz), 9.06 (s, 1H), 13.08 (s, 1H); HPLC Rt=5.63 min. HRMS calcd for C₁₇H₁₃N₅O₂S₁+H 374.0688, found 374.0678. Anal. (C₁₇H₁₃N₅O₂S₁.0.05 TFA) C, H, N, S.

### Example 7

### 2-12-(1,4,5,6-Tetrahydro-pyrimidin-2-yl)-phenylsulfanylmethyl]-1H-benzimidazole-4-carboxylic Acid Amide (7)

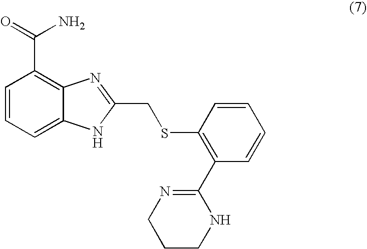

¹H NMR (DMSO-d₆) δ 1.94-2.04 (m, 2H), 2.63-2.55 (m, 2H), 2.95-3.02 (m, 2H) 3.74 (s, 2H), 7.35-7.40 (m, 1H), 7.48-7.90 (m, 7H), 8.06 (d, 1H, J=7.4 Hz), 8.23 (d, 1H, J=7.4 Hz), 8.74 (br s, 1H); HPLC Rt=5.55 min. HRMS calcd for C₁₉H₁₉N₅O₁S₁+H 366.1389, found 366.1396. Anal. (C₁₉H₁₉N₅O₁S₁.2.1 TFA) C, H, N, S.

### Example 8

### 2-(4-Methoxy-phenylsulfanylmethyl)-1H-benzimidazole-4-carboxylic Acid Amide (8)

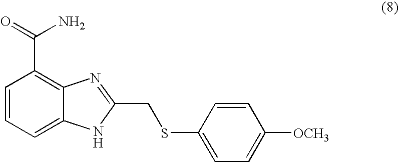

IR (KBr) 3431, 1680, 1664, 1209, 1147, 1037 cm⁻¹; ¹H NMR (DMSO-d₆) δ 3.71 (s, 3H), 4.4 (s, 2H), 6.85-6.88 (m, 2H), 7.28-7.37 (m, 3H), 7.65-7.86 (m, 3H), 8.64 (br s, 1H), 11.03 (br s, 1H); HPLC Rt=5.72 min. HRMS calcd for C₁₆H₁₅N₃O₂S₁+H 314.0963, found 314.0952. Anal. (C₁₆H₁₅N₃O₂S₁.1.9 TFA) C, H, N, S.

### Example 9

### 2-(4-Acetylamino-phenylsulfanylmethyl)-1H-benzimidazole-4-carboxylic Acid Amide (9)

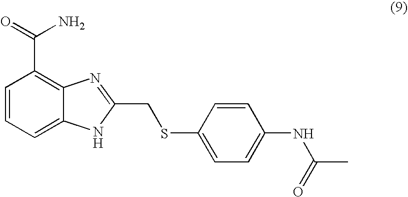

IR (KBr) 3450, 3383, 1730, 1674, 1559, 1198, 1134 cm⁻¹; ¹H NMR (DMSO-d₆) δ 1.99 (s, 3H), 4.51 (s, 2H), 7.26-7.98 (m, 8H), 8.69 (br s, 2H), 10.00 (br s, 1H); HPLC Rt=4.64 min. HRMS calcd for C₁₇H₁₆N₄O₂S₁+H 341.1072, found 341.1064. Anal. (C₁₇H₁₆N₄O₂S₁.0.75H₂O, 1.1 TFA) C, H, N, S.

### Example 10

### 2-(4-Amino-phenylsulfanylmethyl)-1H-benzimidazole-4-carboxylic Acid Amide (10)

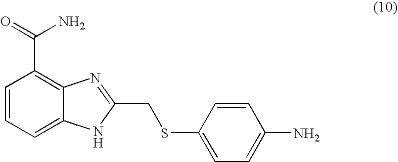

IR (KBr) 3439, 1730, 1670, 1634, 1554, 1495, 1199, 1140 cm⁻¹; ¹H NMR (DMSO-d₆) δ 4.43 (s, 2H), 6.70-6.88 (m, 3H), 7.09-7.56 (m, 4H), 7.70-8.05 (m, 4H), 8.65 (br s, 1H); HPLC Rt=4.04 min. HRMS calcd for C₁₅H₁₄N₄O₁S₁+H 299.0967, found 299.0959. Anal. (C₁₅H₁₄N₄O₁S₁.0.5 H₂O.1.9 TFA) C, H, N, S.

### Example 11

### 2-Heptylsulfanylmethyl-1H-benzimidazole-4-carboxylic Acid Amide (11)

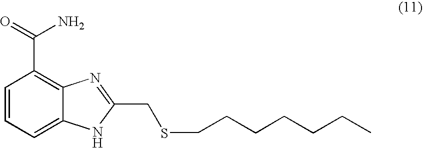

¹H NMR (DMSO-d₆) δ 1.26-1.30 (m, 3H), 1.64-1.84 (m, 8H), 1.96-2.07 (m, 2H), 3.03-3.08 (m, 2H), 4.51 (s, 2H), 7.21-7.27 (m, 2H), 7.72-7.77 (m, 11H), 8.14 (d, 1H, J=8.2 Hz), 8.38 (d, 1H, J=8.1 Hz) 9.57 (br s, 1H); HPLC Rt=6.56 min. HRMS calcd for C₁₅H₂₃N₄O₃S₁+Na 328.1460 found 328.1449

### Example 12

### 2-(4-Dimethylamino-phenylsulfanylmethyl)-1H-benzimidazole-4-carboxylic Acid Amide (12)

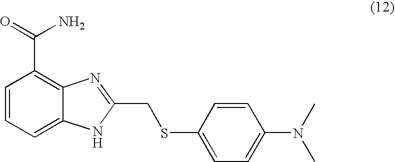

¹H NMR (DMSO-d₆) δ 2.91 (s, 6H), 4.28 (s, 2H), 6.62 (d, 2H, J=8.8 Hz), 6.72 (m, 1H), 7.24-7.31 (m, 3H), 7.66 (d, 1H, J=7.7 Hz), 7.95 (d, 1H, J=7.7 Hz), 9.33 (br s, 1H), 11.83 (br s, 1H); HPLC Rt=4.55 min. HRMS calcd for C₁₇H₁₈N₄O₁S, 326.1201 found 326.1194. Anal. (C₁₇H₁₈N₄O₁S₁.0.7 H₂O) C, H, N, S.

### Example 13

### 2-(4-Trifluoromethyl-phenylsulfanylmethyl)-1H-benzimidazole-4-carboxylic Acid Amide (13)

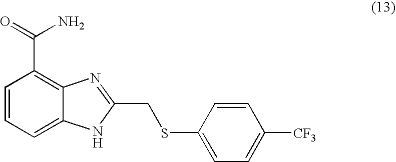

¹H NMR (DMSO-d₆) δ 4.72 (s, 2H), 7.29-7.35 (m, 1H), 7.69-7.75 (m, 7H), 7.84 (d, 1H, J=7.4 Hz), 13.06 (br s, 1H); HPLC Rt=5.98 min. MS calcd for C₁₆H₁₂F₃N₃O₁S₁+H 352 found 352. Anal. (C₁₆H₁₂F₃N₃O₁S₁.0.4 H₂O) C, H, N, S.

### Example 14

### 2-(4-Methylsulfanyl-phenylsulfanylmethyl)-1H-benzimidazole-4-carboxylic Acid Amide (14)

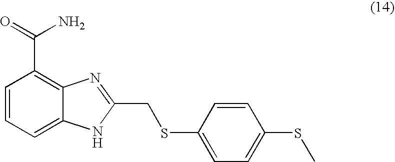

¹H NMR (DMSO-d₆) δ 2.42 (s, 3H), 4.47 (s, 2H), 7.16-7.28 (m, 4H), 7.36 (d, 2H, J=7.7 Hz), 7.60-7.66 (m, 2H), 7.77 (d, 1H, J=7.4 Hz), 12.92 (br s, 1H); HPLC Rt=5.15 min. HRMS calcd for C₁₆H₁₅N₃O₁S₂+H 330.0735 found 330.0749. Anal. (C₁₆H₁₅N₃O₁S₂.0.2 H₂O) C, H, N, S.

### Example 15

### 2-11(2-Hydroxy-benzyl)-methyl-amino]-methyl)-1H-benzimidazole-4-carboxylic Acid Amide (15)

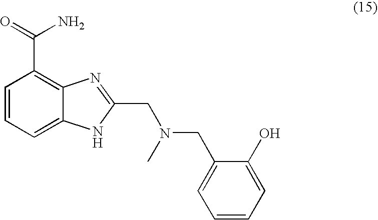

IR (KBr) 3379, 1726, 1659, 1601, 1252, 756 cm⁻; ¹H NMR (DMSO-d₆) δ 2.77 (s, 3H), 4.43 (s, 2H,), 4.70 (s, 2H,), 6.86 (t, 2H, J=7.4 Hz), 6.99 (d, 1H, J=8.5 Hz), 7.27 (t, 1H, J=7.7 Hz), 7.37 (t, 1H, J=7.7 Hz), 7.45 (d, 1H, J=7.0 Hz), 7.73 (s, 1H), 7.85 (d, 1H, J=7.7 Hz), 7.90 (d, 1H, J=7.7 Hz), 8.65 (s, 1H), 10.44 (s, 1H); HPLC Rt=4.73 min. HRMS calcd for C₁₇H₁₈N₄O₂+H 311.1508, found 311.1508. Anal. (C₁₇H₁₈N₄O₂.0.7 H₂O) C, H, N.

### Example 16

### 2-[(3-Pyrrolidin-1-yl-propylamino)-methyl]-1H-benzimidazole-4-carboxylic Acid Amide (16)

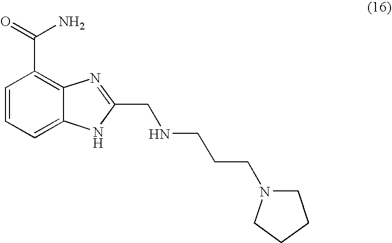

¹H NMR (DMSO-d₆) δ 1.97-2.10 (m, 6H), 3.53-3.79 (m, 8H), 4.56 (m, 2H), 7.30-7.38 (m, 1H), 7.68 (m, 1H), 7.78-7.91 (m, 3H), 9.50 (m, 1H), 9.91 (m, 1H); HPLC Rₜ 3.90 min. HRMS calcd for C₁₆H₂₃N₅O₁+H 302.1981, found 302.1976. Anal. (C₁₆H₂₃N₅O₁.2.5 TFA) C, H, N.

### Example 17

### 2-[4-(4-Acetyl-phenyl)-piperazin-1-vimethyl]-1H-benzimidazole-4-carboxylic Acid Amide (17)

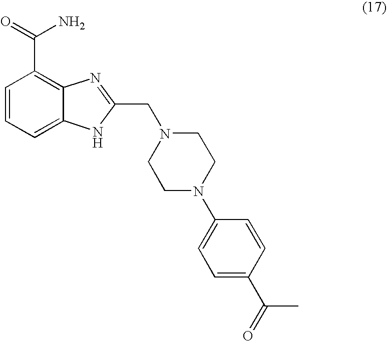

¹H NMR (DMSO-d₆) δ 2.46 (m, 3H), 3.63-3.66 (m, 8H), 4.68 (m, 2H), 5.17 (m, 1H), 7.04 (d, 2H, J=8.8 Hz), 7.31-7.38 (m, 1H), 7.64-7.73 (m, 1H), 7.79-7.91 (m, 4H), 8.53 (br s, 1H); HPLC Rt=4.62 min. HRMS calcd for C₂₁H₂₃N₅O₂+H 378.1930, found 378.1940. Anal. (C₂₁H₂₃N₅O₂.2.0 TFA) C, H, N, S.

### Example 18

### 2-[(Methyl-phenethyl-amino)-methyl]-1H-benzimidazole-4-carboxylic Acid Amide (18)

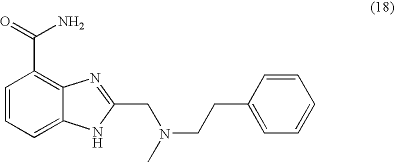

¹H NMR (DMSO-d₆) δ 2.96 (s, 3H), 3.37-3.44 (m, 2H), 3.57-3.68 (m, 2H), 4.74 (s, 2H), 7.23-7.41 (m, 6H), 7.72-7.95 (m, 3H), 10.4 (br s, 1H), 12.85 (br s, 1H); HPLC Rt=5.18 min. HRMS calcd for C₁₈H₂ON₄O₁+H 309.1725, found 309.1715. Anal. (C₁₈H₂ON₄O₁.1.25 TFA) C, H, N.

### Example 19

### 2-(3,4-Dihydro-1H-isoqluinolin-2-ylmethyl)-1H-benzimidazole-4-carboxylic Acid Amide (19)

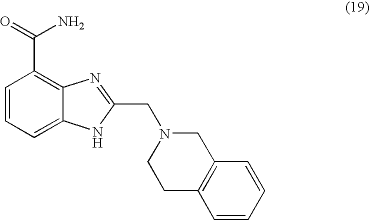

IR (KBr) 3412, 1670, 1615, 1198, 1138, 1020 cm⁻¹; ¹H NMR (DMSO-d₆) δ 3.08-3.18 (m, 2H), 3.58-3.68 (m, 2H), 4.53 (s, 2H), 4.79 (s, 2H), 7.17-7-38 (m, 5H), 7.68-7.95 (m, 4H), 8.6 (br s, 1H); HPLC Rt=4.83 min. HRMS calcd for C₁₈H₁₈N₄O₁+H 307.1559, found 307.1569. Anal. (C₁₈H₁₈N₄O₁.2 H₂O.0.9 TFA) C, H, N.

### Example 20

### 2-[(Methyl-phenyl-amino)-methyl]-1H-benzimidazole-4-carboxylic Acid Amide (20)

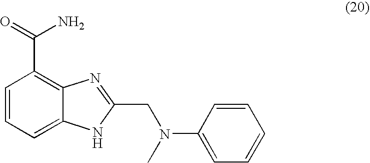

IR (KBr) 3395, 1669, 1601, 1506, 1199, 1138 cm⁻¹; ¹H NMR (DMSO-d₆)63.12(s, 3H), 4.96 (s, 2H), 6.68-6.86 (m, 3H), 7.15-7.22 (m, 2H), 7.41-7.49 (m, 1H), 7.74-7.99 (m, 4H), 8.72 (br s, 1H); HPLC Rt=4.92 min. MS calcd for C₁₆H₁₆N₄O₁+H 1281, found 281. Anal. (C₁₆H₁₆N₄O₁.1.0 H₂O.1.5 TFA) C, H, N.

### Example 21

### 2-[(1-Aza-bicyclo[2.2.2] oct-3-ylamino)-methyl]-1H-benzimidazole-4-carboxylic Acid Amide (21)

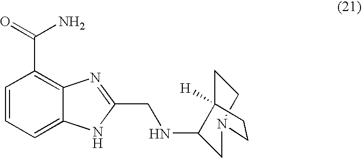

IR (KBr) 3400, 1678, 1601, 1502, 1203, 1132 cm⁻¹; ¹H NMR (DMSO-d₆)δ 1.91-2.10 (m, 4H), 2.28 (m, 1H), 3.38-4.01 (m, 6H), 4.1 (s, 2H), 7.32-46 (m, 1H), 7.63-7.76 (m, 1H), 7.81-7.96 (m, 2H), 8.18-8.36 (m, 2H), 8.57 (br s, 1H), 12.97 (br s, 1H); HPLC Rt=3.49 min. MS calcd for C₁₆H₂₁N₅O₁+H 300, found 300. Anal. (C₁₆H₂₁N₅O₁.0.25 H₂O.2.3 TFA) C, H, N.

### Example 22

### 2-(3-Amino-pyrrolidin-1-ylmethyl)-1H-benzimidazole-4-carboxylic Acid Amide (22)

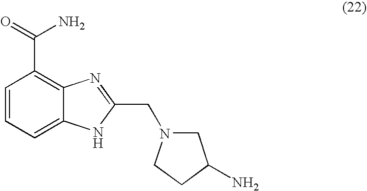

¹H NMR (DMSO-d₆) δ 2.59-2.64 (m, 4H), 3.22-3.32 (m, 3H), 4.50 (m 2H), 4.89 (s, 11H), 7.47-7.52 (m, 11H), 7.79 (br s, 11H), 7.91 (d, 11H, J=8.1 Hz), 7.98 (d, 11H, J=7.7 Hz), 8.56 (br s, 1H), 9.23 (br s, 2H); HPLC Rt=3.66 min. HRMS calcd for C₁₃H₁₇N₅O₁+H 262.1668 found 262.1662. Anal. (C₁₃H₁₇N₅O₁.1.5 H₂O2.6 HCl) C, H, N.

### Example 23

### 2-{[Methyl-(2-methylamino-ethyl)-amino]-methyl}-1H-benzimidazole-4-carboxylic Acid Amide (23)

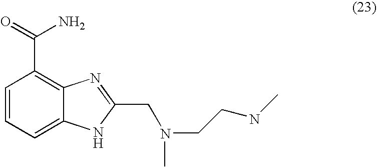

¹H NMR (DMSO-d₆) δ 2.52 (s, 6H), 3.44-3.57 (m, 2H), 3.69-3.81 (m, 2H), 3.94-4.03 (m, 1H), 4.81 (s, 2H), 7.35-7.40 (m, 1H), 7.71 (m, 1H), 7.84 (d, 1H, J=8.1 Hz), 7.90 (d, 1H, J=7.7 Hz), 8.59 (br s, 2H); HPLC Rt=3.58 min. HRMS calcd for C₁₃H₁₉N₅O₁+H 260.1511 found 260.1505. Anal. (C₁₃H₁₉N₅O₁.1.5 H₂O₂O.2.5 HCl) C, H, N.

### Example 24

2-(4-Propionylamino-phenylsulfanylmethyl)-1H-benzimidazole-4-carboxylic Acid Amide (24)

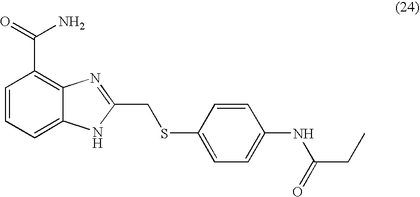

Resin-bound 2-(4-amino-phenylsulfanylmethyl)-1H-benzoimidazole-4-carboxylic acid amide (2.0 g, from Example 10), in 5% 2,4,6-collidine/CH₂Cl₂ (20 mL), was acylated with 0.47 g of propionyl chloride (5.1 mmol). The reaction was shaken on a wrist-action shaker for 1 hr, and then filtered, washed and dried in vacuo for 12 hr. Cleavage was accomplished by treatment with 20 mL of 95% TFA/H₂O for 2 hr. The reaction was filtered and the resin rinsed with additional TFA/H₂O. The combined filtrates was reduced in vacuo and purified by flash silica gel chromatography using a solvent system (7.5% acetone/CH₂Cl₂) to give 111 mg of a white amorphous solid.

¹H NMR (DMSO-d₆) δ 1.05 (t, 3H, J=7.5 Hz), 2.29 (d, 2H, J=7.5 Hz), 4.44 (m, 2H), 7.26 (t, 1H, J=7.8 Hz), 7.35 (d, 2H, J=8.4 Hz), 7.52 (d, 2H, J=8.7 Hz), 7.65-7.68 (m, 2H), 7.78 (d, 1H, J=7.5 Hz), 9.07 (br s, 1H), 9.88 (br s, 1H), 12.82 (br s, 1H); HPLC Rt=5.00 min. MS calcd for C₁₈H₁₈N₄O₂S₁+H 355 found 355. Anal. (C₁₈H₁₈N₄O₂S₁.0.6 H₂O, 0.2 EtOAc) C, H, N, S.

The compounds of Examples 25 and 26 were prepared in a manner similar to Example 24, except with variation of the acid halide R⁶C(O)-halide, e.g.:

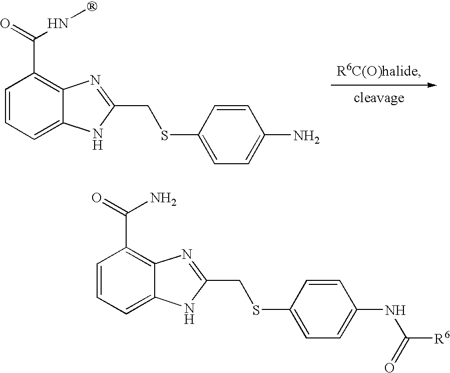

### Example 25

### 14-(4-Carbamoyl-1H-benzoimidazol-2-ylmethylsulfanyl)-phenyl]-carbamic Acid Benzyl Ester (25)

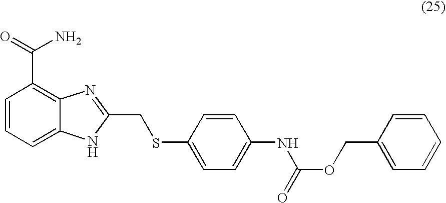

¹H NMR (DMSO-d₆) δ 4.41 (s, 2H), 5.12 (s, 2H), 7.19-7.42 (m, 9H), 7.58-7.66 (m, 2H), 7.77 (d, 1H, J=7.5 Hz), 9.81 (br s, 1H), 10.10 (br s, 1H); HPLC Rt=6.42 min. MS calcd for C₁₈H₁₈N₄O₄S₁+H 433 found 433. Anal. (C₁₈H₁₈N₄O₄S₁.0.3 EtOAc) C, H, N, S.

### Example 26

### 14-(4-Carbamoyl-1H-benzoimidazol-2-ylmethylsulfanyl)-phenyl]-carbamic Acid Methyl Ester (26)

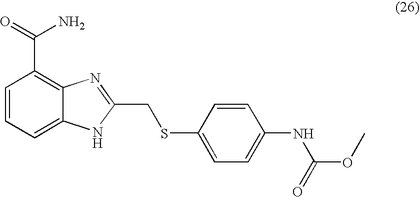

Utilizing the procedure to prepare 2-(4-propionylamino-phenylsulfanylmethyl)1H-benzimidazole-4-carboxylic acid described in Example 24, 4-aminothiophenol treated resin was reacted with methyl chloroformate. This did not yield the expected monoacylated product; instead, bisacylation occurred to produce 4-carbamoyl-2-(4-methoxycarbonylaminophenylsulfanylmethyl)-benzimidazole-1-carboxylic acid methyl ester as confirmed by ¹H NMR and MS.

¹H NMR (DMSO-d₆) δ 3.65 (s, 3H), 4.07 (s, 3H), 4.63 (s, 2H), 7.31-7.36 (m, 4H), 7.39-7.42 (m, 1H), 7.74 (br s, 1H), 7.93-7.96 (m, 2H), 8.10-8.13 (m, 1H) 8.65 (br s, 1H), 9.67 (br s, 1H); HPLC Rt=7.38 min. MS calcd for C₁₉H₁₈N₄O₅S₁+Na 437 found 437.

The above product was dissolved in 10 mL of methanol containing 0.50 mL of 50% aqueous NaOH. This solution was stirred for 2 min, diluted with 20 mL of water and acidified to pH 5 with 1N HCl. The aqueous layer was extracted with EtOAc (×3). The organic layer was dried (MgSO₄), filtered and concentrated. The residue was purified by flash silica gel chromatography (5% CH₃OH/CH₂Cl₂) to give 76.3 mg of the title product as a white amorphous solid.

¹H NMR (DMSO-d₆) δ 3.63 (s, 3H), 4.41 (s, 2H), 7.24 (t, 1H, J=7.8 Hz), 7.29-7.34 (m, 4H), 7.62-7.71 (m, 2H), 7.78 (d, 1H, J=7.5 Hz), 9.10 (br s, 1H), 9.68 (br s, 1H), 12.88 (br s, 1H); HPLC Rt=5.14 min. MS calcd for C₁₇H₁₆N₄O₃S₁+Na 379 found 379. Anal. (C₁₇H₁₆N₄O₃S₁.0.2H₂O.0.3 EtOAc) C, H, N, S.

### Example 27

### 2-(4-Nitro-benzenesulfonylmethyl)-1H-benzimidazole-4-carboxylic Acid Amide (27)

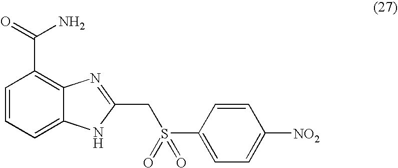

To a solution of 2-(4-nitro-phenylsulfanylmethyl)-1H-benzimidazole-4-carboxylic acid amide (0.10 g, 0.30 mmol) in 1.5 mL methanol at 0° C. was added 0.24 g potassium peroxomonosulfate (0.39 mmol) in 1.5 mL H₂O. The reaction was stirred at 0° C. until complete (by HPLC analysis). It was then diluted with water and the pH was adjusted to 5 with 1N NaOH. The aqueous sample was extracted with EtOAc (×3). The organic layer was dried (MgSO₄), filtered and the solvent removed in vacuo. The residue was purified by flash silica gel chromatography (5% MeOH/EtOAc) to give 17.1 mg (15.6%) of the sulfone (a white amorphous solid).

IR (KBr) 3412, 1662, 1601, 1533, 1350, 1309, 1161 cm⁻¹; ¹H NMR (DMF-d₇)δ 5.43 (s, 2H), 7.36-7.42 (m, 1H), 7.52-7.57 (m, 1H), 7.79-7.83 (m, 1H), 7.94-7.80 (m, 1H), 8.21 (d, 2H, J=7.7 Hz), 8.53 (d, 2H, J=7.7 Hz), 8.75 (br s, 1H), 13.28 (br s, 1H); HPLC Rt=5.71 min. HRMS calcd for C₁₅H₁₂N₄O₅S₁+H361.0607, found 331.0598. Anal. (C₁₅H₁₂N₄O₅S₁.0.25 EtOAc) C, H, N, S.

### Example 28

### 2-(4-Nitro-benzenesulfinylmethyl)-1H-benzimidazole-4-carboxylic Acid Amide (28)

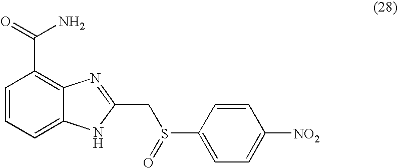

From the final reaction of Example 27, Compound 28 was isolated as a minor product.

IR (KBr) 3346, 3225, 1668, 1604, 1523, 1344, 1037 cm⁻¹; ¹H NMR (DMF-d₇) δ 4.70-4.99 (m, 2H), 7.34-7.39 (m, 1H), 7.57 (m, 1H), 7.76 (d, 1H, J=7.1 Hz), 7.93-7.99 (m, 3H), 8.44 (d, 2H, J=8.5 Hz), 8.97 (br s, 1H), 13.13 (br s, 1H); HPLC Rt=5.71 min. HRMS calcd for C₁₅H₁₂N₄O₄S₁+H 345.0658, found 345.0650. Anal. (C₁₅H₁₂N₄O₄S₁.0.4 H₂O-0.1 EtOAc.0.2 Hexane) C, H, N, S.

The compounds of Examples 29-38 were prepared in a manner as described in Example 27 for oxidation of the corresponding sulfanyl compound:

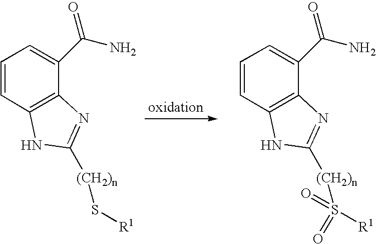

### Example 29

### 2-(4-Hydroxy-benzenesulfonylmethyl)-1H-benzimidazole-4-carboxylic Acid Amide (29)

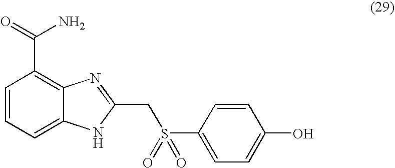

¹H NMR (DMSO-d₆) δ 4.11 (br s, 1H), 5.06 (s, 2H), 6.91-6.99 (m, 2H), 7.38 (br s, 1H), 7.60-7.70 (m, 3H) 7.79-7.83 (m, 1H), 7.87-7.91 (m, 1H), 8.67 (br s, 1H), 10.72 (br s, 1H); HPLC Rt=4.75 min. MS calcd for C₁₅H₁₃N₃O₄S₁+Na 354 found 354. Anal. (C₁₅H₁₃N₃O₄S, 0.5H₂O) C, H, N, S.

### Example 30

### 2-(2-Butylamino-ethanesulfonylmethyl)-1H-benzimidazole-4-carboxylic Acid Amide (30)

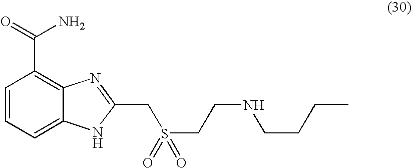

¹H NMR (DMSO-d₆) δ 0.85-0.91 (m, 3H), 1.24-1.37 (m, 2H), 1.48-1.59 (m, 2H), 2.93-2.99 (m, 2H), 3.38-3.44 (m, 2H), 3.73-3.78 (m, 2H), 4.74 (br s, 1H), 5.12 (s, 2H), 7.30-7.36 (m, 1H), 7.71 (br s, 1H), 7.79 (d, 1H, J=7.7 Hz), 7.85 (d, 1H, J=7.7 Hz) 8.65 (br s, 2H); HPLC Rt=4.49 min. HRMS calcd for C₁₅H₂₂N₄O₃S₁+Na 339.1491 found 339.1502. Anal. (C₁₅H₂₂N₄O₃S₁.1.1 HCl.0.9 TFA) C, H, N, S.

### Example 31

### 2-(Heptane-1-sulfonylmethyl)-1H-benzimidazole-4-carboxylic Acid Amide (31)

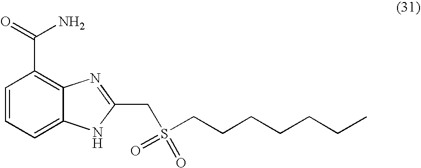

¹H NMR (DMSO-d₆) δ 0.84-0.89 (m, 3H), 1.28-1.48 (m, 8H), 1.79-1.85 (m, 2H), 3.25-3.30 (m, 2H), 4.85 (s, 2H), 6.88 (br s, 1H), 7.34-7.40 (m, 1H), 7.81 (d, 1H, J=8.1 Hz), 7.97-8.01 (m, 1H), 9.08 (br s, 1H), 12.04 (br s, 1H); HPLC Rt=6.66 min. HRMS calcd for C₁₆H₂₃N₃O₃S₁+Na 360.1358 found 360.1349. Anal. (C₁₆H₂₃N₃O₃S]) C, H, N, S.

### Example 32

### 2-(4-Acetylamino-benzenesulfonylmethyl)-1H-benzimidazole-4-carboxylic Acid Amide (32)

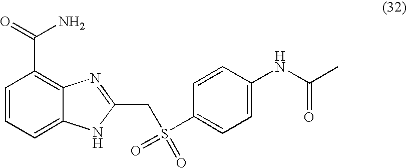

¹H NMR (DMSO-d₆) δ 3.36 (s, 3H), 5.03 (s, 2H), 7.27-7.35 (m, 1H), 7.59 (br s, 1H), 7.67-7.83 (m, 7H), 10.39 (br s, 1H), 13.08 (br s, 1H); HPLC Rt=4.79 min. HRMS calcd for C₁₇H₁₆N₄O₄S₁+H 373.0971 found 373.0982. Anal. (C₁₇H₁₆N₄O₄S₁.0.5 H₂O1.1 CH₃OH)C, H, N, S.

### Example 33

### 2-(4-Methoxy-benzenesulfonylmethyl)-1H-benzimidazole-4-carboxylic Acid Amide (33)

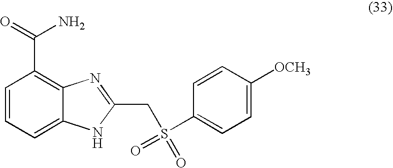

¹H NMR (DMSO-d₆) (mixture of rotamers, 15H) δ 3.85 (s), 5.03 (s), 7.09-7.13 (m), 7.22 (t, 1H, J=7.7 Hz), 7.33 (t, J=7.7 Hz) 7.56 (br s), 7.65-7.74 (m), 7.81 (d, J=7.7 Hz), 8.17 (br s), 8.71 (br s), 12.37 (br s), 13.08 (br s); HPLC Rt=5.37 min. HRMS calcd for C₁₆H₁₅N₃O₄S₁+H 346.0862 found 346.0854. Anal. (C₁₆H₁₅N₃O₄S₁.0.1H₂O.0.1 EtOAc) C, H, N, S.

### Example 34

### 2-(4-Trifluoromethyl-benzenesulfonylmethyl)-1H-benzimidazole-4-carboxylic Acid Amide (34)

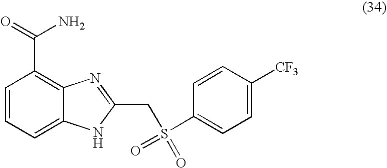

¹H NMR (DMSO-d₆) (mixture of rotamers, ¹²¹I) δ 5.23-5.26 (m), 7.19-7.23 (m), 7.31-7.36 (m), 7.59 (br s), 7.73 (d, J=7.7 Hz), 7.81 (d, J=7.0 Hz), 8.02 (br s), 8.18 (br s), 8.31 (br s), 8.62 (br s), 12.47 (br s), 13.6 (br s); HPLC Rt=6.30 min. HRMS calcd for C₁₆H₁₂F₃N₃O₃S₁+H 384.0630 found 384.0640. Anal. (C₁₆H₁₂F₃N₃O₃S₁) C, H, N, S.

### Example 35

### 2-(4-Methanesulfonyl-benzenesulfonylmethyl)-1H-benzimidazole-4-carboxylic Acid Amide (35)

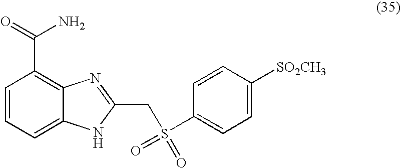

¹H NMR (DMSO-d₆) (mixture of rotamers, 15H) δ 2.53 (s), 5.24-5.28 (m), 7.19-7.38 (m), 7.59 (m), 7.73-7.83 (m), 8.06-8.18 (m), 8.62 (br s), 13.18 (br s.); HPLC Rt=5.13 min. HRMS calcd for C₁₆H₁₅N₃O₄S₁+H 394.0531 found 394.0539. Anal. (C₁₆H₁₅N₃O₅S₂.0.4 H₂O)C, H, N, S.

### Example 36

### 2-(4-Propionylamino-benzenesulfonylmethyl)-1H-benzimidazole-4-carboxylic Acid Amide (36)

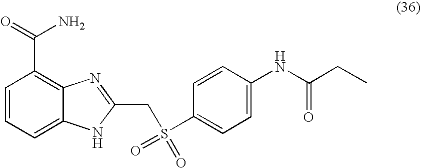

¹H NMR (DMSO-d₆)(mixture of rotamers, 18H) δ 1.08 (t, J=7.5 Hz), 2.37 (d, J=7.5 Hz), 5.03 (m), 7.21 (t, J=7.8 Hz), 7.33 (t, J=7.8 Hz), 7.54-7.59 (m), 7.68-7.83 (m), 8.16 (br s), 10.30 (br s), 12.48 (br s), 13.06 (br s); HPLC Rt=5.23 min. HRMS calcd for C₁₈H₁₈N₄O₄S₁+H 387.1127 found 387.1136. Anal. (C₁₈H₁₈N₄O₄S₁.0.3H_{200.2} EtOAc) C, H, N, S.

### Example 37

### [4-(4-Carbamoyl-1H-benzoimidazol-2-ylmethanesulfonyl)-phenyl]-carbamic Acid Methyl Ester (37)

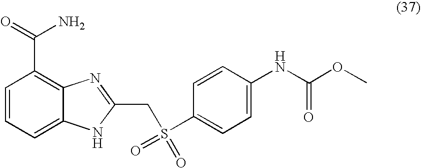

¹H NMR (DMSO-d₆) (mixture of rotamers, 16H) δ 3.70 (s), 5.02 (s), 7.19-7.24 (m), 7.30-7.35 (m), 7.54-7.74 (m), 7.81 (d, J=7.5 Hz), 8.16 (br s), 8.76 (br s), 10.19 (br s), 12.38 (br s), 13.07 (br s); HPLC Rt=5.23 min. HRMS calcd for C₁₇H₁₆N₄O₅S₁+H 389.0920 found 389.0931. Anal. (C₁₇H₁₆N₄O₅S₁.0.5H₂O.0.2 EtOAc) C, H, N, S.

### Example 38

### [4(4-Carbamoyl-1H-benzoimidazol-2-ylmethanesulfonyl)-phenyl]-carbamic Acid Benzyl Ester (38)

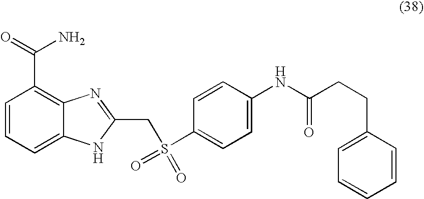

¹H NMR (DMSO-d₆) (mixture of rotamers, 20H) δ 5.02 (s), 5.18 (s), 7.19-7.24 (m), 7.30-7.46 (m), 7.54-7.77 (m), 7.81 (d, 1H, J=7.6 Hz), 8.16 (br s), 8.78 (br s), 10.31 (br s), 12.38 (br s), 13.06 (br s); HPLC Rt=6.66 min. HRMS calcd for C₂₃H₂ON₄O₅S₁+H 465.1233 found 465.1242. Anal. (C₂₃H₂ON₄O₅S₁.0.27 H₂O.0.2 acetone) C, H, N, S.

### Example 39

### Preparation of 2-(4-Ureido-benzenesulfonylmethyl)-1H-benzoimidazole-4-carboxylic Acid Amide (39)

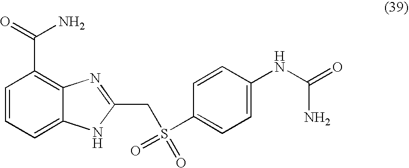

Resin-bound 2-(4-amino-phenylsulfanylmethyl)-1H-benzoimidazole-4-carboxylic acid amide (2.0 g, from Example 10) and 1 mL trimethylsilylisocyanate were heated to 70° C. in 5% DIEA/DMF (20 mL) for 16 hours. The resin was washed, filtered and cleaved with 95% TFA/water (20 mL) for 2 hr. The cleavage cocktail was filtered, and the filtrate was reduced in vacuo. The crude product was cleaned up by silica gel filtration (10% MeOH/CH₂Cl₂) to give 20 mg of a brown solid. This solid was oxidized by the method described in Example 27, which gave after purification by flash silica gel chromatography (10% MeOH/CH₂Cl₂) 10.6 mg of a brown solid.

¹H NMR (DMSO-d₆) (mixture of rotamers, 17H) δ 5.04 (s), 5.18 (s), 7.27-7.32 (m), 7.57-7.82 (m), 8.73 (br s), 9.06 (br s), 10.44 (br s), 12.37 (br s), 13.08 (br s). HPLC Rt=4.80 min. MS calcd for C₁₇H₁₆N₄O₅S₁+Na 396 found 396. Anal.(C₁₇H₁₆N₄O₅S₁.0.2 H₂O.0.35 TFA) C, H, N, S.

### Example 40

### 2-Benzylsulfanyl-1H-benzimidazole-4-carboxylic Acid Amide (40)

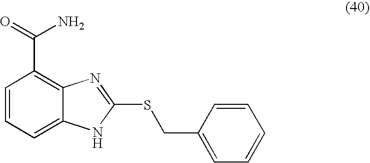

(a) 2-Mercapto-1H-benzimidazole-4-carboxylic Acid Methyl Ester

2-Amino-3-nitro-benzoic acid methyl ester (1.02 g, 5.20 mmol) was hydrogenated utilizing 0.02 g 10% Pd/C and a hydrogen balloon in 50 mL of MeOH for 3 hrs. After this time Celite® was added and the reaction was filtered through an additional pad of Celite®. The solution was concentrated in vacuo and co-evaporated twice with benzene to remove any remaining MeOH. The resulting crude orange/red solid was dissolved in 50 mL dry DMF, to which was added 1.38 g of 1,1′-thiocarbonyldiimidazole (7.74 mmol). After stirring overnight, the reaction was concentrated in vacuo and purified by flash silica gel chromatography using a gradient solvent system (40% CH₂Cl₂/Hex to 5%/35%/60% MeOH/CH₂Cl₂/Hex to 10% MeOH/CH₂Cl₂). The resulting material was contaminated with imidazole; therefore it was washed with 0.1N HCl and water to give 0.90 g (4.32 mmol, 83%) of product as a tan solid.

¹H NMR (DMSO-d₆) δ 3.91 (s, 3H), 7.24 (t, 1H, J=7.7 Hz), 7.38 (d, 1H, J=7.7 Hz), 7.66 (dd, 1H, J=-1.1, 7.7 Hz), 12.33 (br s, 1H) 12.89 (br s, 11H). HPLC Rt=5.67 min. MS calcd for C₉H₈N₂O₂S+H 209, found 209.

(b) 2-Benzylsulfanyl-1H-benzimidazole-4-carboxylic Acid Amide (40)

Sodium hydride (60% dispersion in mineral oil, 47 mg, 1.18 mmol) was suspended in 2 mL DMF at 0° C. To this was added 165 mg of 2-mercapto-1H-benzimidazole-4-carboxylic acid methyl ester (0.79 mmol) in 2 mL DMF via canula. After rinsing with an additional 2 mL DMF the reaction was stirred for 10 minutes, at which time 115 μL of benzyl bromide (0.97 mmol) was added via syringe. The reaction was stirred overnight, with warming to 23° C. After quenching with sat. NH₄Cl the solvent was removed by evaporation. The resulting crude solid was dissolved in 50 mL water and extracted with EtOAc (×3). The organic layer was dried (MgSO₄), filtered and concentrated. The material was filtered through a plug of silica gel utilizing 5% Et₂O/CH₂Cl₂ as eluent and taken on to the next step.

The methyl ester was converted to the amide using the method described by Jagdman et al. (Synth. Commun. (1990) 20:1203-1208), with use of 6 equivalents of sodium methoxide, to give 95 mg of product (0.33 mmol, 41% overall) as a white solid.

IR (KBr) 3443, 3148, 3080, 3003, 2960, 2987, 1660, 1597, 1579, 1512, 1467, 1404, 1244, 976, 752, 706 cm⁻¹. ¹H NMR (acetone-d₆)864.69 (s, 2H), 6.84 (br s, 1H), 7.22-7.36 (m, 4H), 7.51-7.55 (m, 2H), 7.58 (dd, 1H, J=1.1, 8.1 Hz), 7.95 (dd, 1H, J=1.1, 7.7 Hz), 9.31 (br s, 1H), 11.91 (br s, 1H). HPLC Rt=6.137 min. HRMS calcd for C₁₅H₁₂N₃OS+Na 306.0677, found 306.0669. Anal. (C₁₅H₁₃N₃OS) C, H, N, S.

### Example 41

### 2-Phenylmethansulfonyl-1H-benzimidazole-4-carboxylic Acid Amide (41)

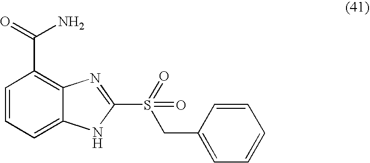

The sulfide of Example 40 was oxidized to the sulfone by treatment with excess 0.1M KMnO4 (aqueous solution in acetone).

¹H NMR (DMSO-d₆) δ 3.31 (s, 2H), 7.28-7.32 (m, 5H), 7.50-7.55 (m, 1H), 7.72-7.83 (m, 1H), 7.90 (br s, 1H), 8.01 (d, 1H, J=7.4 Hz), 8.69 (br s, 1H), 14.31 (br s, 1H). HPLC Rt=5.989 min. HRMS calcd for C₁₅H₁₃N₃O₃S+H 316.0756, found 316.0766.

### Example 42

### 2-(4-Acetylamino-benzylsulfanyl)-1H-benzimidazole-4-carboxylic Acid Amide (42)

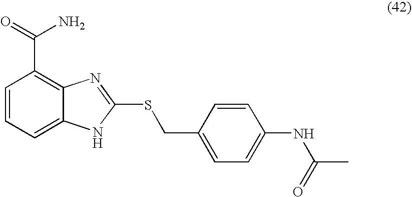

(a) 2-(4-Acetylamino-benzylsulfanyl)-1H-benzimidazole-4-carboxylic Acid Methyl Ester

A solution containing 85 mg of 2-mercapto-1H-benzimidazole-4-carboxylic Acid methyl ester (0.40 mmol) in 4 mL DMF was cooled to 0° C. To this was added 130 mg CsCO₃ (0.40 mmol) followed by 91 mg of 4-acetamidobenzyl chloride (0.49 mmol). The reaction was stirred overnight, allowing it to warm to 23° C., and then concentrated in vacuo. The resulting crude material was suspended in pH 7 buffer and extracted with EtOAc (×3). The combined organic layers were dried (MgSO₄), filtered and concentrated. Purification by flash silica gel chromatography using a gradient solvent system (80% EtOAc/Hex to 100% EtOAc) gave 112 mg of product (0.31 mmol, 77%) as a colorless oil that crystallized upon standing.

IR (KBr) 3283, 3196, 1732, 1709, 1666, 1602, 1547, 1514, 1448, 1431, 1412, 1350, 1302, 1297, 1203, 1145, 1124, 754 cm⁻¹. ¹H NMR (CDCl₃) δ 2.16 (s, 3H), 3.97 (s, 3H), 4.56 (s, 2H), 7.23-7.29 (m, 1H), 7.36-7.47 (m, 4H), 7.82 (d, 1H, J=7.7 Hz), 7.87 (d, 1H, J=7.7 Hz), 10.16 (br s, 1H), not seen 1H(NH). HPLC Rt=5.643 min. Anal. (C₁₈H₁₇N₃O₃S) C, H, N, S.

(b) 2-(4-Acetylamino-benzylsulfanyl)-1H-benzimidazole-4-carboxylic Acid Amide (42)

The methyl ester was converted to the amide using the procedure described in Example 40 to give 57 mg of product (0.16 mmol, 64%) as a light yellow solid.

¹H NMR (500 MHz, DMSO-d₆) δ ¹H NMR (DMSO-d₆) δ 2.01 (s, 3H), 4.56 (s, 2H), 7.24 (t, 1H, J=7.7 Hz), 7.35-7.62 (m, 5H), 7.70-7.82 (m, 2H), 9.09 (br s, 1H), 9.95 (br s, 1H), 13.05 (br s, 1H). HPLC Rt=5.006 min. Anal. (C₁₇H₆N₄O₂S) C, H, N, S.

### Example 43

### 2-(4-Methoxy-benzylsulfanyl)-1H-benzimidazole-4-carboxylic Acid Amide (43)

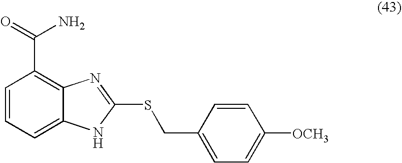

(a) 2-(4-Methoxy-benzylsulfanyl)-1H-benzimidazole-4-carboxylic Acid Methyl Ester

2-Mercapto-1H-benzimidazole-4-carboxylic acid methyl ester was alkylated with 4-methoxybenzyl chloride using the procedure described in Example 42 to give 125 mg of product (0.38 mmol, 84%) as a white solid.

IR (KBr) 3310, 2951, 2930, 2833, 1682, 1610, 1514, 1460, 1435, 1348, 1304, 1253, 1242, 1209, 1176, 1145, 1035, 823, 754, 742 cm⁻¹. ¹H NMR (CDCl₃) δ 3.79 (s, 3H), 3.97 (s, 3H), 4.56 (s, 2H), 6.85 (d, 2H, J=8.8 Hz), 7.23-7.29 (m, 1H), 7.36 (d, 2H, J=8.8 Hz), 7.82 (d, 1H, J=7.7 Hz), 7.88 (d, 1H, J=8.1 Hz), 10.08 (br s, 1H). HPLC Rt=6.670 min. Anal. (C₁₇H₁₆N₂O₃S) C, H, N, S.

(b) 2-(4-Methoxy-benzylsulfanyl)-1H-benzimidazole-4-carboxylic Acid Amide (43)

The methyl ester was converted to the amide using the procedure described in Example 40 to give 57 mg of product (0.16 mmol, 64%) as a light yellow solid.

¹H NMR (DMSO-d₆) (mixture of rotamers, 15H) δ 3.33 (s), 3.71 (s), 4.50 (s), 4.56 (s), 6.87 (d, J=8.6 Hz), 7.16 (t, J=7.8 Hz), 7.24 (t, J=7.8 Hz), 7.38 (d, J=8.6 Hz), 7.45 (br s), 7.55 (dd, J=0.7, 7.8 Hz), 7.63 (d, J=7.6 Hz), 7.68 (d, J=7.8 Hz), 7.73 (br s), 7.78 (dd, J=0.7, 7.6 Hz), 7.95 (br s), 8.07 (br s), 9.09 (br s), 12.44 (br s), 13.03 (br s). HPLC Rt=5.859 min. MS calcd for C₁₆H₁₀N₃O₂S+H 314, found 314. Anal. (C₁₆H₁₀N₃O₂S.0.5 H₂O) C, H, N, S.

### Example 44

### 2-(4-Nitro-benzylsulfanyl)-1H-benzimidazole-4-carboxylic Acid Amide (44)

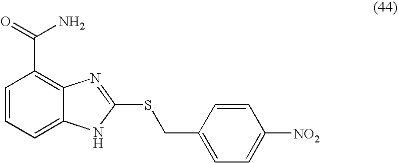

(a) 2-(4-Nitro-benzylsulfanyl)-1H-benzimidazole-4-carboxylic Acid Methyl Ester

2-Mercapto-1H-benzimidazole-4-carboxylic acid methyl ester was alkylated with 4-nitrobenzyl bromide using the procedure described in Example 42 to give 122 mg of product (0.35 mmol, 77%) as a light yellow solid.

IR (KBr) 3396, 2953, 1693, 1599, 1520, 1489, 1454, 1431, 1344, 1317, 1265, 1205, 1145, 1032, 860, 756, 707, 567 cm⁻¹. ¹H NMR (CDCl₃) δ 3.98 (s, 3H), 4.67 (s, 2H), 7.25-7.31 (m, 1H), 7.63 (d, 2H, J=8.8 Hz), 7.81-7.89 (m, 2H), 8.16 (d, 2H, J=8.8 Hz), 10.18 (br s, 1H). HPLC Rt=7.177 min. Anal. (C₁₆H₁₃N₃O₄S) C, H, N, S.

(b) 2-(4-Nitro-benzylsulfanyl)-1H-benzimidazole-4-carboxylic Acid Amide (44)

2-(4-Nitro-benzylsulfanyl)-1H-benzimidazole-4-carboxylic acid methyl ester (78 mg, 0.23 mmol) was heated to reflux overnight in 10 mL 4:2:4 water/1,4-dioxane/conc. HCl. After cooling the reaction mixture to 0° C., the resulting white precipitate was filtered off, rinsed with cold water and dried under vacuum. The carboxylic acid was characterized by HPLC and MS (Rt=6.126 min, calcd for C₁₅H₁₁N₃O₄S+H 330, found 330). The acid was converted to the acid chloride by refluxing in 5 mL of thionyl chloride overnight. The crude acid chloride, after removal of excess reagent in vacuo, was suspended in 5 mL THF and added to a solution of 100 μL of NH₄OH in 10 mL 9:1 THF/water at 0° C. After stirring 2 hr, the reaction was poured into brine and extracted with EtOAc (×3). The organic layer was dried (MgSO₄), filtered and concentrated. The crude material was purified by semi-preparative reverse phase HPLC to give 9 mg of product (0.027 mmol, 11%) as an off-white solid.

¹H NMR (500 MHz, DMSO-d₆) (mixture of rotamers, 12H) δ 4.68 (s), 4.74 (s), 7.16 (t, J=7.8 Hz), 7.23 (t, J=7.8 Hz), 7.63-7.77 (m), 8.07 (s), 8.15-8.20 (m), 8.94 (s), 12.56 (s), 13.11 (s). HPLC Rt=6.293 min. Anal. (C₁₅H₁₂N₄O₃S.1.0 H₂O) C, H, N, S.

### Example 45

### 2-(2-Morpholin-4-yl-ethylsulfanylmethyl)-1H-benzimidazole-4-carboxylic Acid Amide (45)

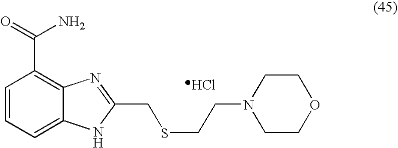

(a) 2-(2-Hydroxy-ethylsulfanylmethyl)-1H-benzimidazole-4-carboxylic Acid Methyl Ester

2-Chloromethyl-1H-benzimidazole-4-carboxylic acid methyl ester was prepared by treatment of 2-chloromethyl-1H-benzimidazole-4-carboxylic acid (Example 1 (a)) with MeOH and HCl. A solution was prepared containing 1.20 g (5.34 mmol) of the 2-chloromethyl-1H-benzimidazole-4-carboxylic acid methyl ester, 2-mercaptoethanol (470 μL, 6.70 mmol), and DIEA (2.0 mL, 11.5 mmol) in 5 mL DMF and stirred overnight. The reaction mixture was concentrated in vacuo and the crude material was purified by flash silica gel chromatography using a gradient solvent system (80% EtOAc/Hex to 100% EtOAc) to give 1.26 g of product (4.73 mmol, 88%) as a tan solid.

¹H NMR (CDCl₃) δ 2.81 (t, 2H, J=5.5 Hz), 3.88 (t, 2H, J=5.5 Hz), 3.93 (s, 1H), 4.00 (s, 3H), 4.08 (s, 2H), 7.25-7.33 (m, 1H), 7.91-7.94 (m, 2H), NH not seen. MS calcd for C₁₂H₁₄N₂O₃S+H 267, found 267.

(b) 2-(2-Chloro-ethylsulfanylmethyl)-1H-benzimidazole-4-carboxylic Acid Methyl Ester

A solution of 1.16 g of 2-(2-hydroxy-ethylsulfanylmethyl)-1H-benzimidazole-4-carboxylic acid methyl ester (4.35 mmol) and 950 μL thionyl chloride (13.0 mmol) in 50 mL CHCl₃ was heated to reflux for 2.5 hr., in the general manner described by Fong et al. (Can. J. Chem. (1979) 57:1206-1213). The reaction was then cooled to 0° C. and quenched by addition of pH 7 phosphate buffer. The aqueous layer was extracted with EtOAc (×3). The combined organic layers were dried (MgSO₄), filtered and concentrated in vacuo. The crude solid isolated was purified by flash silica gel chromatography (50% EtOAc/hexanes) to give 1.23 g of product (4.32 mmol, 99%) as a yellow solid.

¹H NMR (CDCl₃) δ 2.91 (t, 2H, J=7.3 Hz), 3.62 (t, 2H, J=7.3 Hz), 4.02 (s, 3H), 4.07 (s, 2H), 7.29-7.35 (m, 1H), 7.91-7.94 (m, 2H), NH not seen. MS calcd for C₁₂H₁₃ClN₂O₂S+H 285, found 285.

(e) 2-(2-Morpholin-4-yl-ethylsulfanylmethyl)-1H-benzimidazole-4-carboxylic Acid Methyl Ester

2-(2-Chloro-ethylsulfanylmethyl)-1H-benzimidazole-4-carboxylic acid methyl ester (146 mg, 0.51 mmol) and morpholine (500 μL, 5.73 mmol) were heated to 100° C. in 4.5 mL of DMF overnight. The reaction was concentrated in vacuo and the crude material was purified by flash silica gel chromatography using a gradient solvent system 5-10% MeOH/CH₂Cl₂) to give 88 mg of product (0.26 mmol, 51%) as a yellow oil that solidified upon standing.

¹H NMR (CDCl₃) δ 2.38-2.71 (m, 8H), 3.64-3.68 (m, 4H), 4.01 (s, 3H), 4.04 (s, 2H), 7.27-7.33 (m, 1H), 7.88-7.92 (m, 2H), 10.55 (br s, 1H). MS calcd for C₁₆H₂₁N₃O₃S+H 336, found 336.

(d) 2-(2-Morpholin-4-yl-ethylsulfanylmethyl)-1H-benzimidazole-4-carboxylic Acid Amide (45)

The methyl ester was converted to the amide using the procedure described in Example 42 to give the free amine as a very viscous oil.

¹H NMR (free amine, DMSO-d₆) δ 2.25-2.37 (m, 4H), 2.42-2.50 (m, 2H), 2.64-2.77 (m, 2H), 3.48-3.58 (m, 4H), 4.05 (s, 2H), 7.25-7.36 (m, 1H), 7.62-7.87 (m, 3H), 9.19 (br s, 1H), 12.89 (br s, 1H). HPLC Rt=3.886 min. MS calcd for C₁₅H₂ON₄O₂S+H 321, found 321.

The amine was converted to the hydrochloride salt by treatment with 3 equivalents of HCl (4M HCJ in 1,4-dioxane) in Et₂O. The product was isolated by concentration and drying under vacuum for 16 hr. Anal. (C₁₅H₂ON₄O₂S.1.0 HCl.0.5H₂O.0.2 Et₂O) C, H, N, S.

### Example 46

### 2-[2-(3,4-Dihydro-1H-isoquinolin-2-yl)-ethylsulfanylmethyl]-1H-benzoimidazole-4-carboxylic Acid Amide (46)

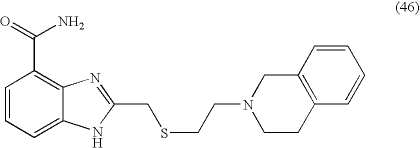

1,2,3,4-Tetrahydroisoquinoline was alkylated with 2-(2-chloroethylsulfanylmethyl)-1H-benzimidazole-4-carboxylic acid methyl ester (146 mg, 0.51 mmol), and the resulting ester was converted to an amide per Example 40 to give 35 mg of the title compound as an off-white solid.

IR (KBr) 3338, 3179, 2927, 2810, 1664, 1654, 1648, 1638, 1617, 1609, 1492, 1419, 1240, 745 cm⁻¹. ¹H NMR (DMSO-d₆) δ 2.60-2.87 (m, 8H), 3.54 (s, 2H), 4.06 (s, 2H), 6.95-7.13 (m, 4H), 7.28 (t, 1H, J=7.6 Hz), 7.60 (d, 1H, J=7.6 Hz), 7.72 (br s, 1H), 7.82 (d, 1H, J=7.6 Hz), 9.22 (br s, 1H), 12.93 (br s, 1H). HRMS calcd for C₂₀H₂₃N₄OS+H 367.1593, found 367.1601. Anal. (C₂OH₂₃N₄OS.0.6 H₂O.0.2 Acetone) C, H, N, S.

### Example 47

### 3,4-Dihydro-1H-2-thia-4a,9-diaza-fluorene-8-carboxylic Acid Amide (47)

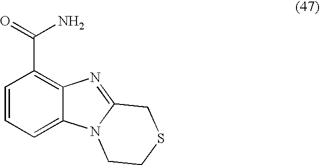

(a) 3,4-Dihydro-1H-2-thia-4a,9-diaza-fluorene-8-carboxylic Acid Methyl Ester (47a)

Compound 47(a) was isolated as a by-product of the reaction used to prepare 2-(220 morpholin-4-yl-ethylsulfanylmethyl)-1H-benzimidazole-4-carboxylic acid amide (Example 45) and 2-[2-(3,4-dihydro-1H-isoquinolin-2-yl)-ethylsulfanylmethyl]-1H-benzoimidazole-4-carboxylic acid amide (Example 46).

IR (KBr) 3546, 3422, 3368, 3233, 1692, 1435, 1308, 1250 1204, 1149, 756 cm⁻¹. ¹H NMR (CDCl₃) δ 3.19 (t, 2H, J=5.7 Hz), 4.03 (s, 3H), 4.21 (s, 2H), 4.38 (t, 2H, J=5.7 Hz), 7.32 (t, 1H, J=7.8 Hz), 7.50 (dd, 1H, J=1.1, 7.8 Hz), 7.98 (dd, 1H, J=1.1, 7.8 Hz). HPLC Rt=4.945 min. HRMS calcd for C₁₂H₁₃N₂O₂S+H 349.0698, found 249.0696.

(b) 3,4-Dihydro-1H-2-thia-4a,9-diaza-fluorene-8-carboxylic Acid Amide (47)

A 195 mg sample of 3,4-dihydro-1H-2-thia-4a,9-diaza-fluorene-8-carboxylic Acid methyl ester (0.79 mmol) was hydrolyzed by refluxing in a mixture of 2:4:3 1,4-dioxane/water/conc. HCl (10 mL) for 3 hr. The crude acid was isolated by concentrating the reaction mixtures and adding pH ˜3-4 sulfate buffer. The acid precipitated out of solution and was collected by filtration (obtained 105 mg, 0.45 mmol). It was then converted to the acid fluoride by treatment with TFFH (220 mg, 0.83) and DIEA (150 μL) in 3 mL CH₃CN overnight. The acid fluoride solution was added to 10 mL of THF containing 500 μL of NH₄OH, which had been cooled to 0° C. After stirring for 1 hr the reaction was concentrated and the resulting solid suspended in water. The product was isolated by filtration and washing (water and Et₂O) to give 48 mg of a tan solid.

¹H NMR (DMSO-d₆) δ 3.29 (t, 2H, J=5.7 Hz), 4.21 (s, 2H), 4.42 (t, 2H, J=5.7 Hz), 7.36 (t, 1H, J=7.8 Hz), 7.73 (dd, 1H, J=1.1, 8.1 Hz), 7.75 (br s, 1H), 7.88 (dd, 1H, J=1.1, 7.7 Hz), 9.08 (br s, 1H). HPLC Rt=4.241 min. Anal. (C₁₁H₁₁N₃OS) C, H, N, S.

### Example 48

### 2-(4-Acetylamino-benzenesulfonylmethyl)-1-methyl-1H-benzoimidazole-4-carboxylic Acid Amide (48)

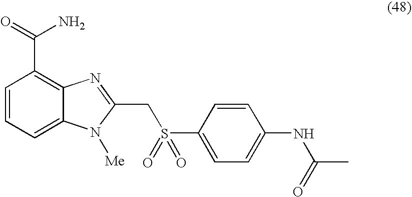

A 51 mg sample of 2-(4-acetylamino-benzenesulfonylmethyl)-1H-benzimidazole-4-carboxylic acid amide (Example 32, 0.13 mmol) was dissolved in 1.3 mL of DMF. To this solution was added 50 mL of DIEA (0.28 mmol) and 15 μL methyl iodide and the mixture was stirred overnight. Two more portions of reagents were added over the next two days. A tan precipitate formed, which was collected by filtration and washed (Et₂O) to give 16 mg (0.04 mmol) of product as a tan solid.

¹H NMR (DMSO-d₆) δ 2.10 (s, 3H), 3.85 (s, 3H), 5.25 (s, 2H), 7.40 (d, 1H, J=7.7 Hz), 7.58 (br s, 1H), 7.66-7.90 (m, 6H), 8.68 (br s, 1H), 10.36 (br s, 1H). HRMS calcd for C₁₈H₁₉N₄O₄S+H 387.1127, found 387.1124. HPLC Rt=5.404 min. Anal. (C₁₈H₁₈N₄O₄S.1.2 H₂O) C, H, N, S.

### Example 49

### 2-14-(2-Hydroxy-acetylamino)-benzenesulfonylmethyl]-1H-benzoimidazole-4-carboxylic Acid Amide (49)

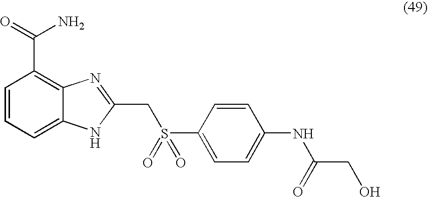

Acetoxyacetic acid (0.211 g, 2.48 mmol), HATU (1.00 g, 2.48 mmol), and DIEA (0.69 mL, 3.96 mmol) was added to resin-bound 2-(4-amino-phenylsulfanylmethyl)-1H-benzoimidazole-4-carboxylic acid amide (1.0 g, 0.62 mmol, from Example 10) in 20 mL DMF. The reaction was shaken in a wrist action shaker for 1 hour at room temperature, filtered, washed and dried under vacuum overnight. The resin was suspended in 10 mL of acetic acid containing 30% aqueous hydrogen peroxide (1 mL). After stirring at room temperature for 16 hours, the resin was filtered, washed and cleaved with 20 mL of 95% TFA/water for 2 hours. The cleavage mixture was filtered and the filtrate reduced in vacuo to give 56.7 mg of a brown solid. This intermediate was dissolved in 4 mL of methanol and treated with K₂CO₃ (200 mg, 1.45 mmol), dissolved in 1 mL of water, for 1 hour. Excess reagent was filtered off and the reaction concentrated. The crude alcohol was purified by silica gel chromatography (5% MeOH/EtOAc) to give 24 mg of product as a tan solid.

¹H NMR (DMSO-d₆)(mixture of rotamers, 17H) δ 3.99-4.05 (m), 5.05 (s), 5.69-5.71 (m), 7.23-7.36 (m), 7.54 (br s), 7.72-7.84 (m), 7.90-7.93 (m), 8.74 (br s), 10.11 (br s), 12.37 (br s), 13.07 (br s). HPLC Rt=4.59 min. HRMS calcd for C₁₇H₁₆N₄O₅S, 389.0920 found 389.0931. Anal. (C₁₇H₁₆N₄O₅S₁.0.5 EtOAc) C, H, N, S.

### Example 50

### 2-(4-Amino-phenylsulfanylmethyl)-1H-benzoimidazole-4-carboxylic Acid Amide (50)

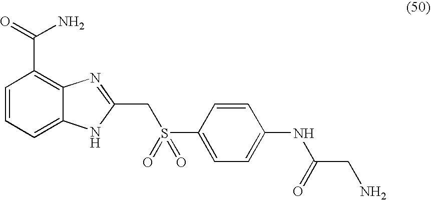

To resin-bound 2-(4-amino-phenylsulfanylmethyl)-1H-benzoimidazole-4-carboxylic acid amide (2.0 g, 0.66 mmol, from Example 10) in 20 mL DMF was added (N-(95 fluorenylmethoxycarbonyl)-glycine (0.589 g, 1.98 mmol), HATU (0.752 g, 1.98 mmol), and DIEA (0.68 mL, 3.96 mmol). After agitating 1 hour at room temperature, the resin was washed, filtered and cleaved with 95% TFA/water (20 mL) for 2 hr. The cleavage cocktail was filtered, and the filtrate was reduced in vacuo. The crude product was cleaned up by silica gel filtration (50% acetone/CH₂Cl₂). This intermediate was oxidized per Example 27 to give 171 mg of a brown solid. The fluorenylmethoxycarbonyl was removed by stirring in 20 mL of CH₂Cl₂ containing 100 μL DBU for 1 hour. Evaporation of the reaction solvent and purification by semipreparatory RP-HPLC chromatography gave 31 mg of product as a brown solid.

¹H NMR (DMSO-d₆) (mixture of rotamers, 17H) δ 3.73 (s), 5.06 (s), 5.18 (s), 7.15 (d, J=7.5 Hz), 7.76 (s), 7.80 (d, J=7.5 Hz), 8.12 (br s), 10.82 (br s), 10.44 (br s); HPLC Rt=4.33 min. MS calcd for C₁₇H₁₇N₅O₄S₁+Na 396 found 396. Anal.(C₁₇H₁₇N₅O₄S₁.0.1 H₂O.0.7 TFA) C, H, N, S.

### Example 51

### 2-[4-(2,5-Dioxo-imidazolidin-1-yl)-benzenesulfonylmethyl]-1H-benzoimidazole-4-carboxylic Acid Amide (51)

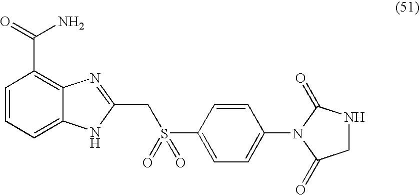

Resin-bound 2-(4-amino-phenylsulfanylmethyl)-1H-benzoimidazole-4-carboxylic acid amide (0.75 g, from Example 10) was acylated with (N-(9-fluorenylmethoxycarbonyl)glycine and the 9-fluorenylmethoxycarbonyl was deprotected as described in Example 50. This resin-bound intermediate 2-[4-(2-amino-acetylamino)-benzenesulfonylmethyl]-1H-benzoimidazole-4-carboxylic acid amide was cyclized by treatment with 4-nitrophenyl chloroformate (0.35 g, 1.74 mmol) and DIEA (0.35 mL, 2.01 mmol) in 10 mL DMF. After shaking for 16 hours the resin was filtered, washed, and cleaved with 95% TFA/water (20 mL). The filtrate was reduced in vacuo and the resulting crude oil purified by preparatory RP-HPLC to give 21 mg of product as a tan solid.

¹H NMR (DMSO-d₆) δ 4.10 (2H, s), 5.18 (2H, s), 7.20 (d, 2H, J=Hz), 7.40 (br s, 1H), 7.59 (br s, 1H), 7.70 (d, 2H, J=8.5 Hz), 7.75 (d, 1H, J=7.9 Hz), 7.82 (d, 1H, J=7.9 Hz), 7.88 (br s, 1H), 7.96 (d, 2H, J=8.5 Hz), 8.51 (br s, 2H). HPLC Rt=4.79 min. MS calcd for C₁₈H₁₅N₅O₅S₁ 414 found 414. Anal.(C₁₈H₁₅N₅O₅S₁.1H₂O.1.4 TFA) C, H, N, S.

### Example 52

### 2-Methylsulfanyl-3H-benzoimidazolc-4-carboxylic Acid Amide (52)

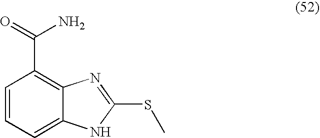

A sample of 2-amino-3-nitrobenzamide (5.00 g, 27.6 mmol) was hydrogenated to the diamine in methanol (300 mL) utilized 10% Pd/C as described in Example 1. The resulting crude diamine was dissolved in 100 mL of DMF, to which was added 5.40 g of 1,1′-thiocarbonyldiimidazole (30.3 mmol). The reaction was stirred for 2 hr, at which time DIEA (7.25 mL, 41.6 mmol) and methyl iodide (2.20 mL, 35.3 mmol) were added. After stirring an additional 1 hr, the reaction was concentrated under vacuum. The crude product was suspended in 1.0M aqueous KH₂PO₄ (500 mL) and placed in a refrigerator overnight. The resulting solid was filtered off, rinsed with water and dried under vacuum to give 5.45 g (26.3 mmol, 95%) of product as a light yellow solid.

¹H NMR (Pyridine-d₅) δ 2.61 (s, 3H), 7.21 (t, 1H, J=7.8 Hz), 7.47-7.56 (m, 1H), 8.28 (br s, 1H), 8.36-8.46 (m, 1), 9.72 (br s, 1H), 14.21 (br s, 1H). HPLC Rt=2.071 min. LR/MS for (C₉H₉N₃OS+H) 208. Anal. (C₉H₉N₃OS.0.1 H₂O) C, H, N.

### Example 53

### 2-Methanesulfonyl-[1H]-benzoimidazole-4-carboxylic Acid Amide (53)

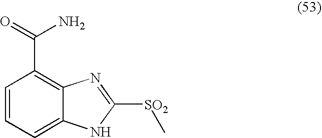

Oxone (7.71 g, 12.5 mmol) in 50 mL of H₂O was added to 2-methylsulfanyl-1H-benzoimidazole-4-carboxylic acid amide (2.00 g; 9.6 mmol) in MeOH (500 mL) at 0° C. The reaction was then allowed to warm to room temperature (RT) and stirred overnight. The solvent was stripped, H₂O was added and the resulting solid was filtered off to give 2.08 g (91%) of product as a tan solid.

IR (KBr) 3414, 1654, 1622, 1603, 1319, 1139 cm. ¹H NMR (DMSO-d₆) δ 3.57 (s, 3H), 7.53 (m, 1H), 7.83 (m, 2H), 7.99 (m, 1H), 8.66 (br s, 1H), 14.38 (br s, 1H). HPLC Rt=2.488 min. LR/MS for (C₉H₉N₃O₃S+H) 240. Anal. (C₉H₉N₃O₃S.0.25 H₂O) C, H, N, S.

### Example 54

### 2-(Methyl-phenethyl-amino)-1H-benzoimidazole-4-carboxylic Acid Amide (54)

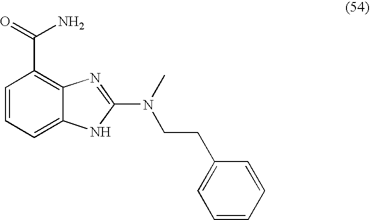

A solution of 2-methanesulfonyl-[1H]-benzoimidazole-4-carboxylic Acid Amide (225 mg, 0.94 mmol) and N-methylphenethylamine (763 mg, 5.65 mmol) in 4 mL diethyleneglycol was heated to 150° C. for 16 hours. After cooling to RT, the crude reaction mixture was purified by preparative HPLC to give 73.7 mg (19%) of product.

IR (KBr) 3373, 3336, 3170, 1686, 1676, 1654 cm⁻. ¹H NMR (DMSO-d₆) δ 2.95 (t, 2H, J=7.2 Hz), 3.16 (s, 3H), 3.83 (t, 2H, J=7.2 Hz), 7.17-7.42 (m, 7H), 7.59-7.65 (m, 2H), 8.62 (br s, 1H), 12.22 (br s, 1H). HPLC Rt=3.216 min. LR/MS for (C₁₇H₁₈N₄O+H) 295. Anal. (C₁₇H₁₈N₄O−0.25H₂O, 1.0 TFA) C, H, N.

The following examples were prepared in a similar manner.

### Example 55

### 2-Methylamino-1H-benzoimidazole-4-carboxylic Acid Amide (55)

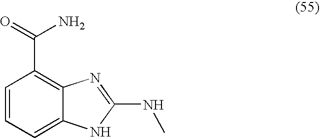

IR (KBr) 3338, 3155, 1686, 1638, 1593 cm⁻¹. ¹H NMR (DMSO-d₆)82.94 (s, 3H), 7.00 (t, 1H, J=7.6 Hz), 7.14 (br s, 1H), 7.32(d, 1H, J=7.6 Hz), 7.46 (br s, 1H), 7.58 (d, 1H, J=7.6 Hz), 8.90 (br s, 1H), 11.68 (br s, 1H). HPLC Rt=2.116 min. LR/MS for (C₉H₁₀N₄O₂+H) 191. Anal. (C₉H₁₀N₄O₂.0.5 H₂O, 0.25 TFA, 0.25 DMF) C, H, N.

### Example 56

### 2-Amino-1H-benzoimidazole-4-carboxylic Acid Amide (56)

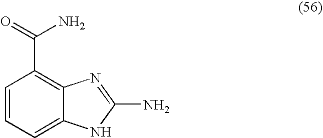

IR (KBr) 3393, 3178, 1655, 1638, 1560 cm⁻¹. ¹H NMR (DMSO-d₆) δ 7.26 (t, 1H, J=7.9 Hz), 7.50 (d, 1H, J=7.9 Hz), 7.70 (br s, 1H), 7.74 (d, 1H, J=7.6 Hz), 8.05 (br s, 2H), 8.32 (br s, 1H), 12.34 (br s, 1H). HPLC Rt=2.117 min. LR/MS for (C₈H₉N₄O+H) 211. Anal. (C₈H₈N₄O-0.3 H₂O, 0.25TFA) C, H, N.

### Example 57

### 2-Dimethylamino-1H-benzoimidazole-4-carboxylic Acid Amide (57)

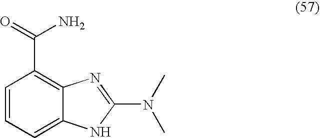

IR (KBr) 3369, 3171, 1675, 1610 cm⁻¹. ¹H NMR (DMSO-d₆) δ 3.2 (s, 6H), 7.21 (t, 1H, J=7.7 Hz), 7.45 (d, 1H, J=7.9 Hz), 7.62 (br s, 1H), 7.66 (d, 1H, J=7.6 Hz), 8.48 (br s, 1H), 12.48 (br s, 1H). HPLC Rₜ 2.370 min. LR/MS for (C₁₀H₁₂N₄O+H) 330. Anal. (C₁₀H₁₂N₄O.1.1 TFA) C, H, N.

### Example 58

### 2-Benzylamino-1H-benzoimidazole-4-carboxylic Acid Amide (58)

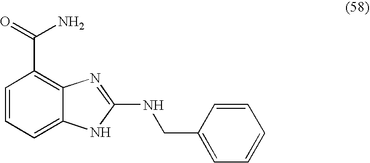

IR (KBr) 3368, 1655, 1630 cm⁻. ¹H NMR (DMSO-d₆) 64.58 (s, 2H), 6.95-7.08 (m, 1H), 7.22-7.64 (m, 8H), 7.79 (br s, 1H), 8.79 (br s, 1H), 11.65 (br s, 1H). HPLC Rt=3.037 min. LR/MS for (C₁₅H₁₄N₄O+H) 267. Anal. (C₁₅H₄N₄O_{0.6} H₂O, 0.25 TFA) C, H, N.

### Example 59

### 2-(2-Diethylamino-ethylamino)-1H-benzoimidazole-4-carboxylic Acid Amide (59)

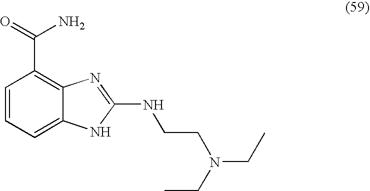

IR (KBr) 3369, 1655, 1638, 1578, 1560 cm⁻. ¹H NMR (DMSO-d₆) δ 0.98 (t, 6H, J=7.4 Hz), 2.52-2.67 (m, 6H), 3.35-3.47 (m, 2H), 6.82 (br s, 1H), 6.93 (t, 1H, J=7.8 Hz), 7.23-7.26 (m, 1H), 7.38 (br s, 1H), 7.50-7.53 (m, 1H), 9.20 (br s, 1H), 11.16 (br s, 1H). HPLC Rt 1.772 min. LR/MS for (C14H₂₁N₄O+H) 275. Anal. (C₁₄H₂₁N₄O_{0.5} H₂O) C, H, N.

### Example 60

### 2-(2-Thiophen-2-yl-ethylamino)-1H-benzoimidazole-4-carboxylic Acid Amide (60)

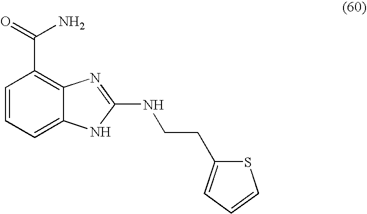

IR (KBr) 3338, 1664, 1560 cm⁻¹. ¹H NMR (DMSO-d₆) δ 3.17 (t, 2H, J=7.0 Hz), 3.71 (t, 2H, J=7.0 Hz), 6.98 (s, 1H), 7.00 (s, 1H), 7.20 (m, 1H), 7.36-7.39 (m, 1H), 7.48-7.54 (m, 1H), 7.68 (br s, 1H), 7.74 (d, 1H, J=7.8 Hz), 7.98 (br s, 1H), 8.34 (br s, 1H), 11.99 (br s, 1H). HPLC Rt=2.964 min. LR/MS for (C₁₄H₁₄N₄OS+H) 287. Anal. (C₁₄H₁₄N₄OS0.5H₂₀, 1.0 TFA) C, H, N.

### Example 61

### 2-[2-(3H-Imidazol-4-yl)-ethylamino]-1H-benzoimidazole-4-carboxylic Acid Amide (61)

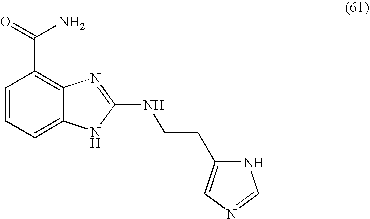

IR (KBr) 3427, 3173, 1655, 1560 cm⁻. ¹H NMR (DMSO-d₆) δ 3.01 (t, 2H, J=6.6 Hz), 3.68-3.76 (m, 2H), 7.17-7.26 (m, 1H), 7.46-7.55 (m, 2H), 7.58-7.76 (m, 2H), 7.98 (br s, 1H), 8.44 (br s, 1H), 8.99 (s, 1H), 11.95 (br s, 1H), 13.95 (br s, 1H). HPLC Rt=2.089 min. LR/MS for (C₁₃H₁₄N₆O+H) 271. Anal.(C₁₃H₁₄N₆O.0.5 H₂O, 2.0 TFA) C, H, N.

PARP Enzyme Inhibition Assay:

The PARP enzyme-inhibiting activities of test compounds were assayed as described by Simonin et al. (J. Biol. Chem. (1993), 268:8529-8535) and Marsischky et al. (J. Biol. Chem. (1995), 270:3247-3254) with minor modifications as follows. Samples (50 μL) containing 20 nM purified PARP protein, 10 μg/mL DNAse I-activated calf thymus DNA (sigma), 500 μM NAD⁺, 0.5 μCi [³²P]NAD⁺, 2% DMSO, and various concentrations of test compounds were incubated in sample buffer (50 mM Tris pH 8.0, 10 mM MgCl₂, 1 mM tris(carboxyethyl)phosphine HCl) at 25° C. for 5 minutes. Under these conditions, the reaction rate was linear for times up to 10 minutes. The reaction was stopped by the addition of an equal volume of ice-cold 40% trichloroacetic acid to the samples, which were then incubated on ice for 15 minutes. The samples were then transferred to a Bio-Dot microfiltration apparatus (BioRad), filtered through Whatman GF/C glass-fiber filter paper, washed 3 times with 150 μL of wash buffer (5% trichloroacetic acid, 1% inorganic pyrophosphate), and dried. [³²P]ADP-Ribose incorporation into the acid-insoluble material was quantitated using a PhosphorImager (Molecular Dynamics) and ImageQuant software. Inhibition constants (Kᵢ) were calculated by non-linear regression analyses using the velocity equation for competitive inhibition (Segel, Enzyme Kinetics: Behavior and Analysis of Rapid Equilibrium and Steady-State Enzyme Systems, John Wiley & Sons, Inc., New York (1975), 100-125). In the case of tight-binding inhibitors, 5 nM enzyme was used and the reaction was incubated at 25° C. for 25 minutes. Kᵢ values for tight-binding inhibitors were calculated using the equation described by Sculley et al. (Biochim. Biophys. Acta (11986), 874:44-53).

Cytotoxicity Potentiation Assay:

A549 cells (ATCC, Rockville, Md.) were seeded into 96-well cell culture plates (Falcon brand, Fisher Scientific, Pittsburgh, Pa.) 16 to 24 hours before experimental manipulation. Cells were then treated with a test compound (or a combination of test compounds where indicated) for either 3 days or 5 days. At the end of treatments, relative cell number was determined either by MTT assay or SRB assay. For the MTT assay, 0.2 μg/μl of MTT (3-(4,5-dimethylthiazol-2-yl)-2,5-diphenyltetrazolium bromide, Sigma Chemical Co., St. Louis, Mo.) was added to each well of a plate, and the plate was incubated in a cell-culture incubator for 4 hours. Metabolized MTT in each well was solubilized in 150 μl of DMSO (Sigma Chemical Co.) with shaking and quantified with a Wallac 1420 Victor plate reader (EG & G Wallac, Gaithersburg, Md.) at 540 nm. For the SRB assay, cells were fixed with 10% trichloroacetic acid (Sigma Chemical Co) for an hour at 4° C. After extensively washing, fixed cells were stained for 30 minutes with 0.4% sulforhodamine B (SRB, Sigma Chemical Co.) in 1% acetic acid (Sigma Chemical Co). Unbound SRB was washed away with 1% acetic acid. Then the cultures were air-dried, and bound dye was solubilized with 10 mM unbuffered Tris base (Sigma Chemical Co) with shaking. The bound dye was measured photometrically with the Wallac Victor plate reader at 515 nm. The ratio of the OD (optical density) value of a compound-treated culture to the OD value of a mock-treated culture, expressed in percentage, was used to quantify the cytotoxicity of a compound. The concentration at which a compound causes 50% cytotoxicity is referred to as IC₅₀. To quantify the potentiation of the cytotoxicity of topotecan or temozolomide by test compounds, a dimensionless parameter PF₅₀ is used and is defined as the ratio of the IC₅₀ Of topotecan or temozolomide alone to the IC₅₀ of topotecan or temozolomide in combination with a test compound. For the compounds of the invention, PF₅₀ values were determined by testing with topotecan.

Inhibition constants (Kᵢ values) and cytotoxicity potentiation parameters (PF₅₀ values) as determined for exemplary compounds of the invention are presented in Table 1 below, where “ND” means not determined.

TABLE 1
PARP Enzyme Inhibition and Cytotoxicity Potentiation

| | | Cytotoxicity |
|---|---|---|
| Compound | Inhibition Constant | Potentiation |
| No. | Kᵢ (nM) | PF₅₀ |
| 1 | 29 ± 7 | 1.2 |
| 2 | 12 | ND |
| 3 | 8 ± 0 | ND |
| 4 | 13 ± 0 | ND |
| 5 | 11 ± 1 | ND |
| 6 | 102 | ND |
| 7 | 6.9 ± 0.4 | 2.2 |
| 8 | 9 ± 1 | ND |
| 9 | 10 ± 3 | 1.1 |
| 10 | 17 ± 1 | ND |
| 11 | 33 ± 6 | ND |
| 12 | 91 ± 3 | ND |
| 13 | 39 ± 5 | ND |
| 14 | 11 ± 2 | ND |
| 15 | 45 ± 3 | ND |
| 16 | 15 ± 3 | ND |
| 17 | 19 ± 2 | 1.2 |
| 18 | 8.6 ± 0.1 | 1.4 |
| 19 | 22 ± 2 | 1.1 |
| 20 | 14 ± 3 | ND |
| 21 | 9 ± 2 | ND |
| 22 | 17 | ND |
| 23 | 14 | ND |
| 24 | 36 ± 2 | ND |
| 25 | 39 ± 0 | ND |
| 26 | 38 ± 1 | ND |
| 27 | 2.5 ± 0.2 | 1.3 |
| 28 | 3.9 ± 0.6 | 1.4 |
| 29 | 24 ± 0 | 1.2 |
| 30 | 26 | 1.1 |
| 31 | 61 ± 10 | ND |
| 32 | 3.8 ± 0.5 | 1.1 |
| 33 | 10.3 ± 0.3 | 1.3 |
| 34 | 12 ± 1 | 1.2 |
| 35 | 5 ± 1 | ND |
| 36 | 17.8 ± 0.8 | ND |
| 37 | 28.5 ± 0.5 | ND |
| 38 | 16 ± 1 | 1.1 |
| 39 | 29.5 ± 2.5 | ND |
| 40 | 70 ± 15 | ND |
| 41 | 800 | ND |
| 42 | 11 ± 3 | 1.1 |
| 43 | 6.5 ± 1.5 | ND |
| 44 | 4.7 ± 0.2 | ND |
| 45 | 48 ± 1 | ND |
| 46 | 33 ± 3 | ND |
| 47 | 93 ± 10 | ND |
| 48 | 10 ± 3 | 1.2 |
| 49 | 7.4 | 1.0 |
| 50 | 34 ± 5 | ND |
| 51 | 24 ± 3 | ND |
| 52 | 27 ± 4 | ND |
| 53 | ND | ND |
| 54 | 35 ± 6 | ND |
| 55 | 85 ± 8 | ND |
| 56 | 319 ± 36 | ND |
| 57 | 44 ± 4 | ND |
| 58 | 66 ± 8 | ND |
| 59 | 23 ± 4 | ND |
| 60 | 81 ± 10 | ND |
| 61 | 12 ± 1 | ND |

While the invention has been described in terms of various preferred embodiments and specific examples, the invention should be understood as not being limited by the foregoing detailed description, but as being defined by the appended claims and their equivalents.

What is claimed is:

1. A compound represented by formula: 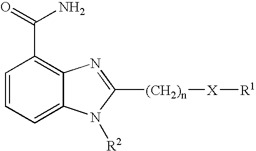
wherein:
n is 0 or 1;
R¹ is H or an alkyl, aryl, heteroaryl, or heterocycloalkyl group unsubstituted or substituted with one or more substituents independently selected from the group consisting of halogens; ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens; ═O; ═S; —CN; —NO₂; alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
X is:
—S(O)ₘ—, wherein m is 0, 1, or 2; or
—N(R³)—, wherein R³ is H or C₁ to C₄ alkyl; or when n=1, —N(R³)— and R¹ together form a 3- to 10-membered heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}R_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more R_{c} groups together cyclize to form part of a heteroaryl or heterocycloalkyl group unsubstituted or substituted with an unsubstituted alkyl group; and
R² is H or alkyl;
or R¹ and R², together with the atoms to which they are bound, form a 5- to 8-membered heterocyclic ring unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR, —C(O)R_{c}, —NR_{c}C(O)NR_{c}R_{c}, —NR_{c}(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
or a pharmaceutically acceptable salt, prodrug, active metabolite, or solvate thereof.

2. A compound, pharmaceutically acceptable salt, prodrug, active metabolite, or solvate thereof according to claim 1, wherein R² is H or lower alkyl.

3. A compound according to claim 2 represented by the formula: 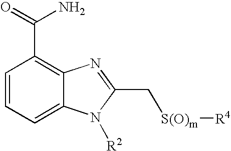
wherein:
R⁴ is hydrogen or an alkyl, aryl, heteroaryl, or heterocycloalkyl group unsubstituted or substituted with one or more substituents selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{z}OR_{c}, —NR_{c}R_{c}, —C(O)NR_{c}, —C(O)OR_{c}, —C(O)R_{c}, —NR_{c}(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
or a pharmaceutically acceptable salt, prodrug, active metabolite, or solvate thereof.

4. A compound, pharmaceutically acceptable salt, prodrug, active metabolite, or solvate thereof according to claim 3, wherein m is 0.

5. A compound, pharmaceutically acceptable salt, prodrug, active metabolite, or solvate thereof according to claim 4, wherein R⁴ is an aryl or heteroaryl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

6. A compound according to claim 5 selected from the group consisting of: 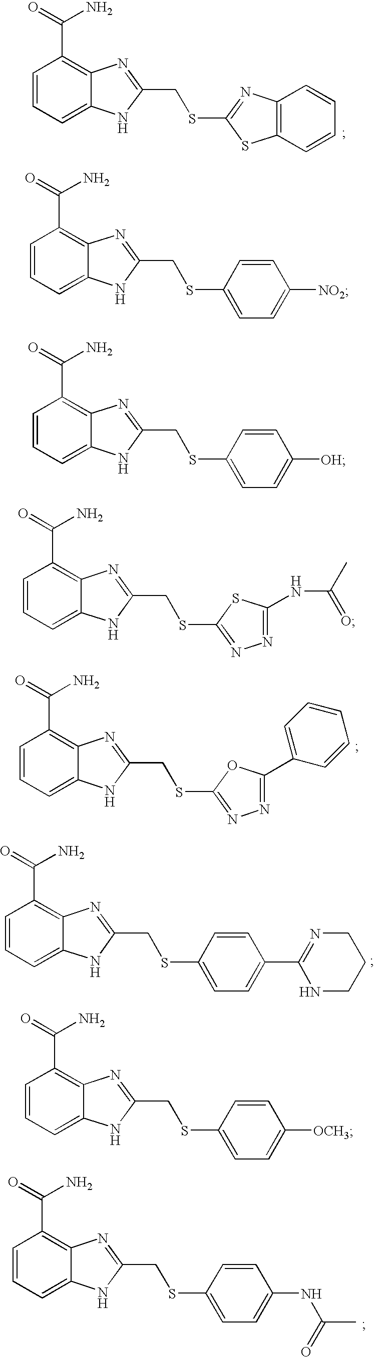 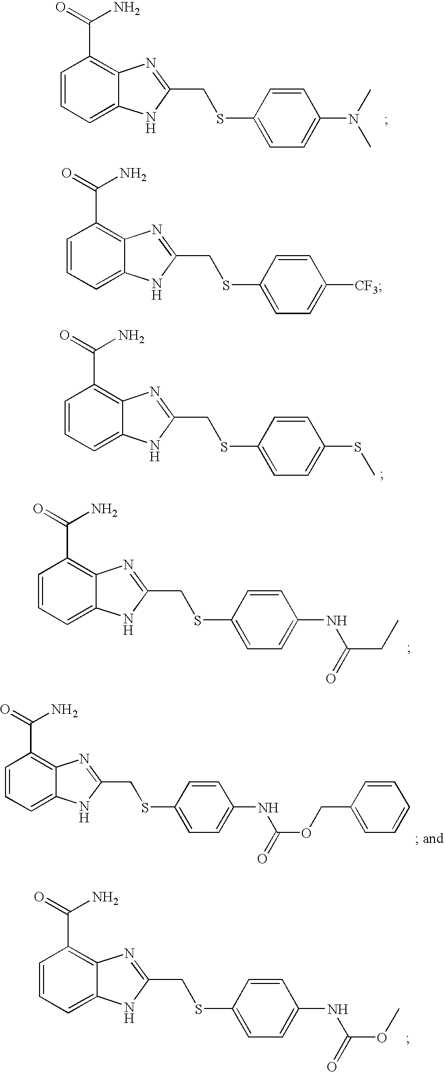
or a pharmaceutically acceptable salt or solvate thereof.

7. A compound, pharmaceutically acceptable salt, prodrug, active metabolite, or solvate thereof according to claim 4, wherein R⁴ is an alkyl group unsubstituted or substituted with one or more substituents selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

8. A compound according to claim 7 selected from the group consisting of: 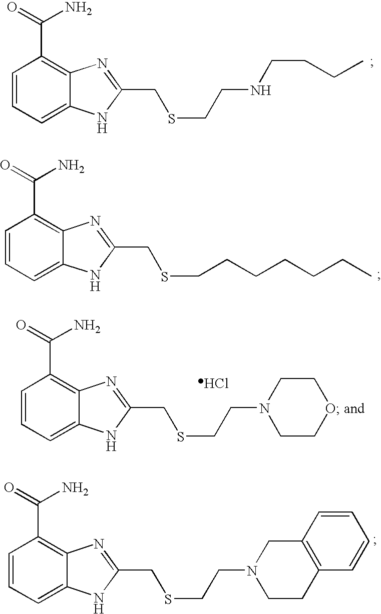
or a pharmaceutically acceptable salt or solvate thereof.

9. A compound, salt, prodrug, metabolite, or solvate according to claim 3, wherein m is 1 or 2.

10. A compound, salt, prodrug, metabolite, or solvate according to claim 9, wherein R⁴ is an aryl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NNHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —N_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

11. A compound according to claim 10 selected from the group consisting of: 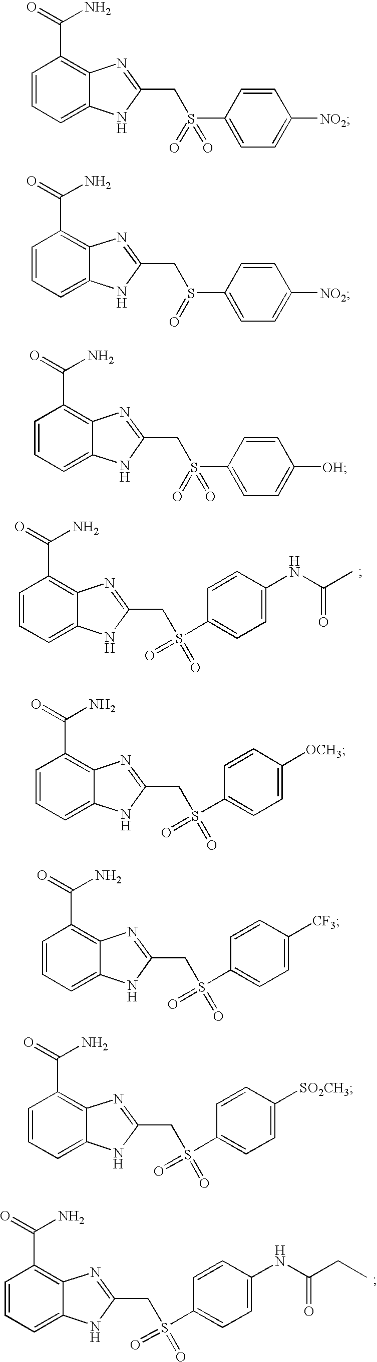 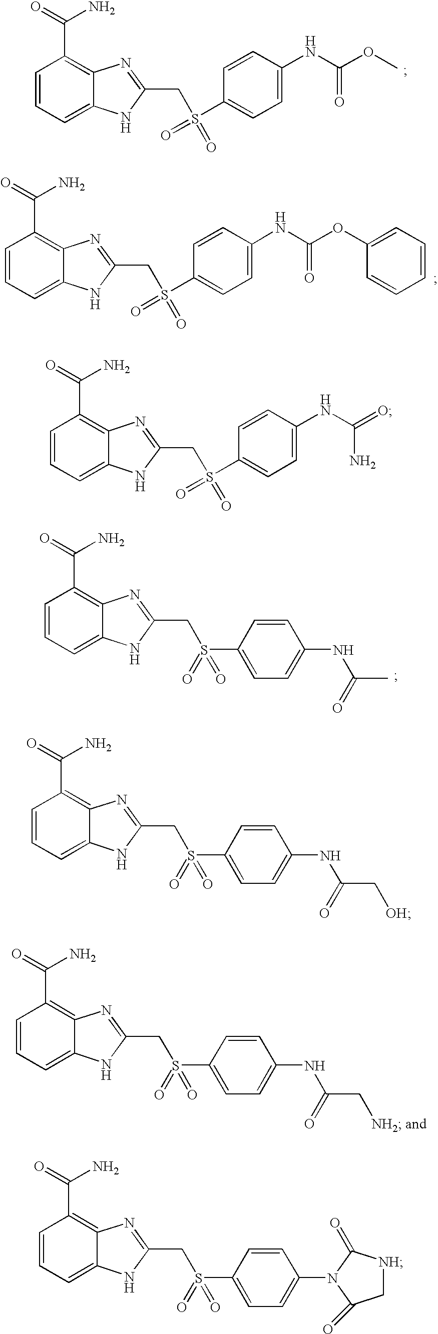
or a pharmaceutically acceptable salt or solvate thereof.

12. A compound, salt, prodrug, metabolite, or solvate according to claim 9, wherein R⁴ is an alkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NNHC(O)NH₂. —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}R_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

13. A compound according to claim 12 selected from the group consisting of: 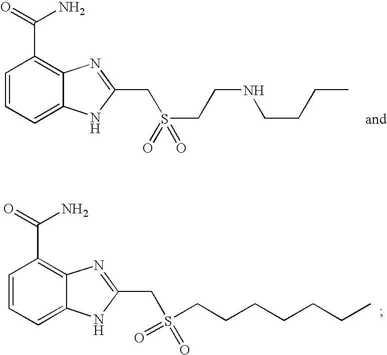
or a pharmaceutically acceptable salt or solvate thereof

14. A compound according to claim 2 having formula: 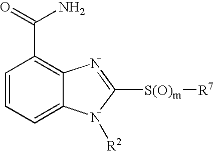
wherein:
R⁷ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, and —NO₂, and alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
or a pharmaceutically acceptable salt or solvate thereof.

15. A compound according to claim 14 selected from the group consisting of: 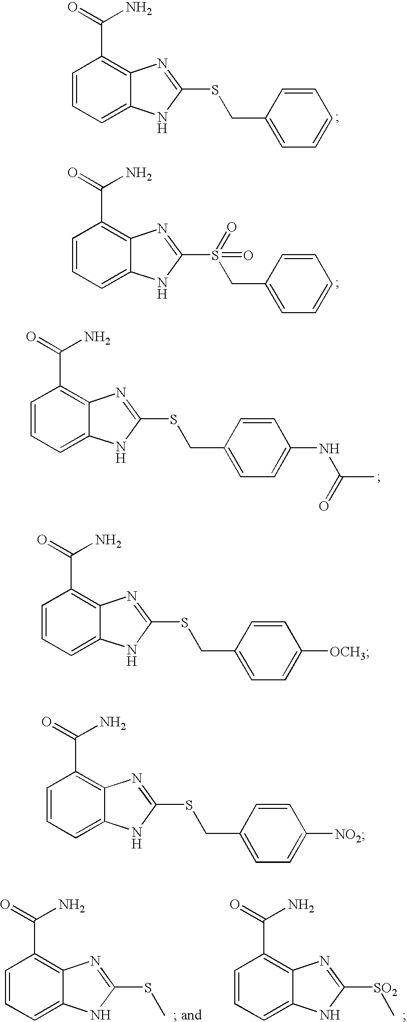
or a pharmaceutically acceptable salt or solvate thereof.

16. A compound according to claim 2 having formula: 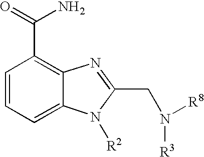
wherein:
R⁸ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHCNH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, and —NO₂, and alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
or R³ and R⁸, together with the atoms to which they are bound, form a 3- to 10-membered heterocyclic ring unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂. —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
or a pharmaceutically acceptable salt, prodrug, active metabolite, or solvate thereof.

17. A compound, salt, prodrug, metabolite, or solvate according to claim 16, wherein:
R³ is H or C₁ to C₄ alkyl; and
R⁸ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂)_{z}—CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

18. A compound according to claim 17 selected from the group consisting of: 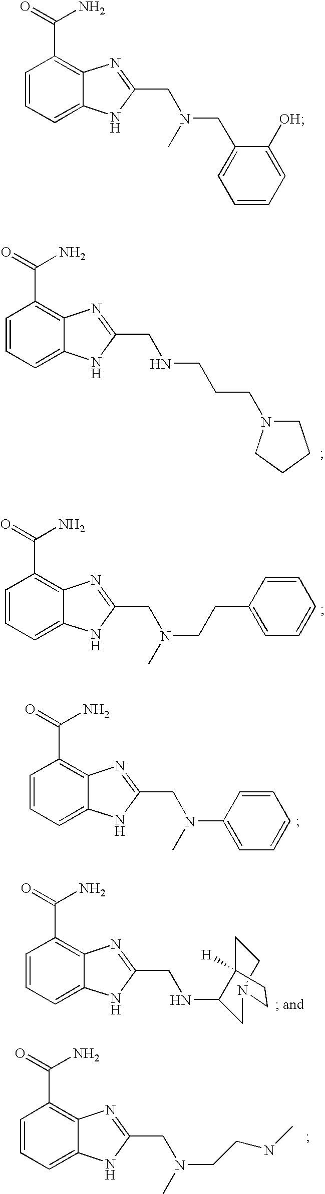
or a pharmaceutically acceptable salt or solvate thereof.

19. A compound, salt, prodrug, metabolite, or solvate according to claim 16, wherein:
R³ and R⁸ together with the atoms to which they are bound form a 3- to 10-membered heterocyclic ring unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1:to 4, ═NH, —NHOH, OH, —C(O)H, —OC(O)H, C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}R_{c}, —NR_{c}R_{c}, —C(O)NR_{c}, —C(O)OR_{c}, —C(O)R_{c}, —NR_{c}C(O)NR_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

20. A compound according to claim 19 selected from the group consisting of: 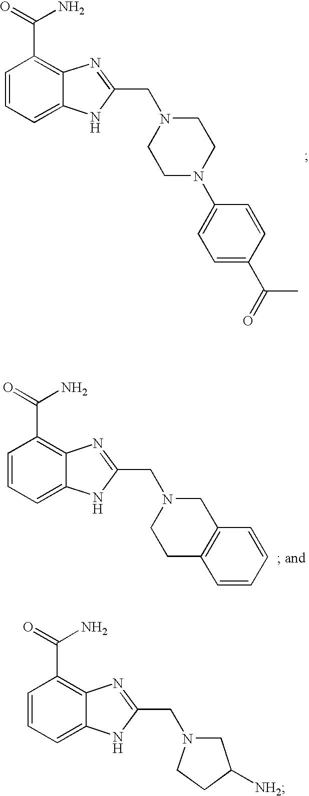
or a pharmaceutically acceptable salt, prodrug, active metabolite, or solvate thereof.

21. A compound, salt, prodrug, metabolite, or solvate according to claim 1, wherein:
R¹ and R², together with the atoms to which they are bound, form a 5- to 8-membered heterocyclic ring unsubstituted or substituted with one or substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group.

22. A compound according to claim 21 of formula: 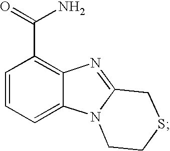
or a pharmaceutically acceptable salt or solvate thereof.

23. A compound, salt, prodrug, metabolite, or solvate according to claim 2 having formula: 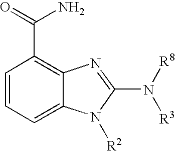
wherein:
R⁸ is an alkyl, aryl, heteroaryl, or heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, and —NO₂, and alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group, or a pharmaceutically acceptable salt, prodrug, active metabolite, or solvate thereof

24. A compound according to claim 23 selected from the group consisting of: 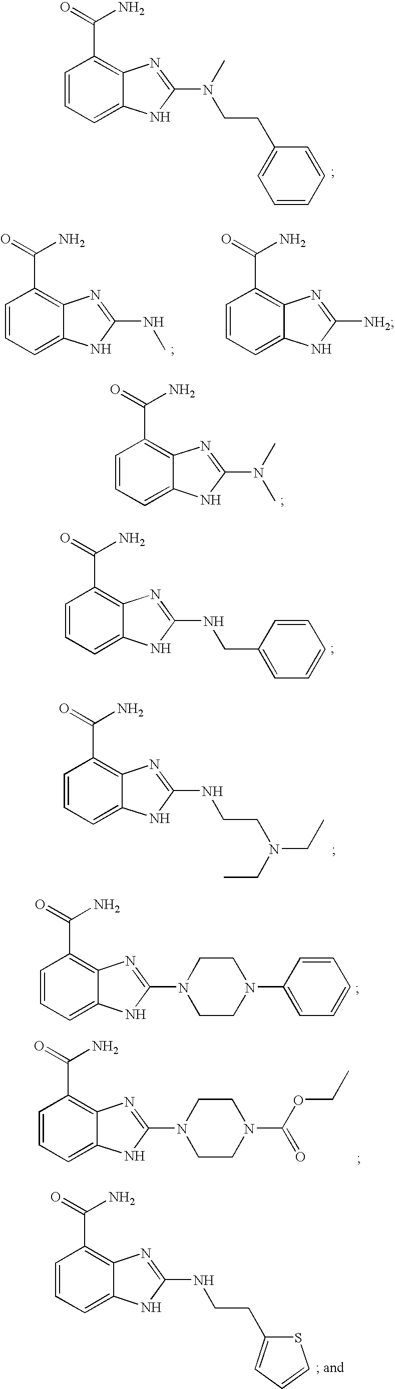 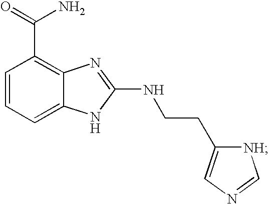
or a pharmaceutically acceptable salt or solvate thereof.

25. A pharmaceutical composition comprising: an effective PARP-inhibiting amount of a compound, salt, prodrug, active metabolite, or solvate defined in claim 1; and a pharmaceutically acceptable carrier therefor.

26. A method of inhibiting PARP enzyme activity comprising: contacting a PARP enzyme with an effective amount of a compound, salt, prodrug, metabolite, or solvate defined in claim 1.

27. A method of inhibiting PARP enzyme activity in mammalian tissue by administering an effective amount of a compound, salt, prodrug, metabolite, or solvate defined in claim 1 to said mammalian tissue.

28. A method of improving the effectiveness of a cytotoxic drug or radiotherapy administered to a mammal in the course of therapeutic treatment, said method comprising: administering to the mammal an effective PARP-inhibiting amount of a compound, salt, prodrug, metabolite, or solvate defined in claim 1 in conjunction with the administration of said cytotoxic drug or radiotherapy.

29. A method for protecting against injury consequent to myocardial ischemia or reperfusion in a mammal comprising: administering to the mammal an effective amount of a compound, salt, prodrug, metabolite, or solvate defined in claim 1.

30. A method for reducing neurotoxicity consequent to a stroke, a head trauma, or a neurodegenerative disease in a mammal comprising: administering to the mammal an effective amount of a compound, salt, prodrug, metabolite, or solvate according to claim 1.

31. A method for delaying the onset of cell senescence associated with skin aging in a mammal comprising: administering to fibroblast cells in the mammal an effective PARP-inhibiting amount of a compound, salt, prodrug, metabolite, or solvate defined in claim 1.

32. A method for preventing the onset of insulin-dependent diabetes in a mammal comprising administering a compound, salt, prodrug, metabolite, or solvate defined in claim 1 to said mammal.

33. A process for synthesizing a compound of formula I: 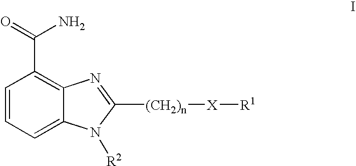
wherein:
n is 0 or 1;
R¹ is H or an alkyl, aryl, heteroaryl, or heterocycloalkyl group unsubstituted or substituted with one or more substituents independently selected from the group consisting of halogens; ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens; ═O; ═S; —CN; —NO₂; alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R_{c} groups together cyclize to form part of a heteroaryl or heterocycloalkyl group unsubstituted or substituted with an unsubstituted alkyl group;
X is:
—S(O)ₘ—, wherein m is 0, 1, or 2; or
—N(R³)—, wherein R³ is H or C, to C₄ alkyl; or —N(R³)— and R¹ together form a 3- to 10-membered heterocycloalkyl group unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)OH, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups, each said group being unsubstituted or substituted with one or more substituents independently selected from the group consisting of halogens, ═O, ═S, —CN, —NO₂, alkyl, alkenyl, alkynyl, aryl, cycloalkyl, heterocycloalkyl, heteroaryl, —(CH₂)_{z}CN where z is an integer from 1 to 4, ═NH, —NHOH, —OH, —C(O)H, —OC(O)H, —C(O)OH, —OC(O)OH, —OC(O)OC(O)H, —OOH, —C(NH)NH₂, —NHC(NH)NH₂, —C(S)NH₂, —NHC(S)NH₂, —NHC(O)NH₂, —S(O₂)H, —S(O)H, —NH₂, —C(O)NH₂, —OC(O)NH₂, —NHC(O)H, —NHC(O)OH, —C(O)NHC(O)H, —OS(O₂)H, —OS(O)H, —OSH, —SC(O)H, —S(O)C(O)OH, —SO₂C(O)OH, —NHSH, —NHS(O)H, —NHSO₂H, —C(O)SH, —C(O)S(O)H, —C(O)S(O₂)H, —C(S)H, —C(SO)OH, —C(SO₂)OH, —NHC(S)H, —OC(S)H, —OC(S)OH, —OC(SO₂)H, —S(O₂)NH₂, —S(O)NH₂, —SNH₂, —NHCS(O₂)H, —NHC(SO)H, —NHC(S)H, and —SH groups unsubstituted or substituted with one or more substituents independently selected from the group consisting of halogens, ═O, —NO₂, —CN, —(CH₂), —CN where z is an integer from 1 to 4, —OR_{c}, —NR_{c}OR_{c}, —NR_{c}R_{c}, —C(O)NR_{c}, —C(O)OR_{c}, —C(O)R_{c}, —NR_{c}C(O)NR_{c}R_{c}, —NR_{c}C(O)R_{c}, —OC(O)OR_{c}, —OC(O)NR_{c}R_{c}, —SR_{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R_{c} is hydrogen, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more R_{c} groups together cyclize to form part of a heteroaryl or heterocycloalkyl group unsubstituted or substituted with an unsubstituted alkyl group; and
R² is H or alkyl;
said process comprising:
providing an electrophilic resin-bound precursor of formula: 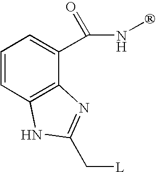
where L is a leaving group and ® represents a support resin;
reacting the electrophilic resin-bound precursor with a nucleophile R¹—X—H, where R¹ and X are as defined above; and
cleaving the product from the resin to yield a compound of the formula I.

34. A method for potentiating the cytotoxicity of a cytotoxic drug or ionizing radiation comprising: contacting cells with an effective amount of a compound, salt, prodrug, metabolite, or solvate defined in claim 1 in combination with the cytotoxic drug or ionizing radiation.

35. A method according to claim 34 wherein the compound, salt, prodrug, metabolite, or solvate has a cytotoxicity potentiation activity corresponding to a PF₅₀ of greater than 1 in a cytotoxicity potentiation assay.

36. A method of treating inflammation comprising: administering an effective amount of a compound, salt, prodrug, metabolite, or solvate defined in claim 1 to a mammal in need of treatment.
